(19) United States
(12) Patent Application Publication
(10) Pub. No.: US 2025/0177353 A1
(43) Pub. Date: Jun. 5, 2025

(54) METHODS OF TREATING MITOCHONDRIA-RELATED DISORDERS

(71) Applicant: Rivus Pharmaceuticals, Inc., Charlottesville, VA (US)

(72) Inventors: Shaharyar Khan, Charlottesville, VA (US); Diane Jorkasky, Devon, PA (US)

(21) Appl. No.: 18/562,560

(22) PCT Filed: May 19, 2022
(86) PCT No.: PCT/US2022/029992

§ 371 Date: Nov. 20, 2023

Related U.S. Application Data
(60) Provisional application No. 63/191,321, filed on May 20, 2021.
(60) Provisional application No. 63/222,841, filed on Jul. 16, 2021.
(60) Provisional application No. 63/307,515, filed on Feb. 7, 2022.

(51) Int. Cl. A61K 31/4168

(52) U.S. Cl. CPC A61K 31/4168

(57) ABSTRACT

Disclosed herein are methods of treatment comprising administering a therapeutically effective amount of 5-[(2,4-dinitrophenoxy)methyl]-1-methyl-2-nitro-1H-imidazole or a pharmaceutically acceptable salt thereof to a subject in need thereof. 5-[(2,4-Dinitrophenoxy)methyl]-1-methyl-2-nitro-1H-imidazole is useful in treating mitochondria-related disorders or conditions including obesity, diabetes, hypertension, cardiovascular disease, and liver diseases.

### CROSS-REFERENCE TO RELATED APPLICATIONS

This application is a 35 USC § 371 National Stage filing of International Application No. PCT/US2022/029992, filed May 19, 2022, which claims the benefit of priority to U.S. Provisional Application No. 63/191,321 filed May 20, 2021, U.S. Provisional Application No. 63/222,841, filed Jul. 16, 2021, and U.S. Provisional Application No. 63/307,515, filed Feb. 7, 2022. The entire contents of the aforementioned applications are incorporated herein by reference in their entireties.

### TECHNICAL FIELD

The present disclosure relates to methods for treating cardiovascular diseases and mitochondria-related disorders or conditions without causing a clinically significant risk of adverse events or overdose.

### BACKGROUND

Cardiovascular diseases are type of diseases that involve the heart and blood vessels, such as coronary artery diseases, hear attack, stroke, heart failure, hypertensive heart disease, and rheumatic heart disease. More than 6.5 million people in the United States have heart failure (Cardiology Today, Apr. 6, 2017). Heart failure with preserved ejection fraction (HFpEF), also known as diastolic heart failure, causes almost one-half of the 6.5 million cases of heart failure in the United States. HFpEF results from abnormalities of active ventricular relaxation and passive ventricular compliance, leading to a decline in stroke volume and cardiac output (Am Fam Physician. 2017 Nov. 1;96 (9): 582-588). In hear failure with reduced ejection fraction (HFrEF), also known as systolic heart failure, the heart muscle is not able to contract adequately and expels less oxygen-rich blood into the body. Mortality was similar between patients with HFpEF and HFrEF (Cardiology Today, Apr. 6, 2017). Heart failure with midrange ejection fraction (HFmrEF) is a new category of heart failure, in between HFpEF and HFrEF. HFmrEF has a prevalence of 10-20% of heart failure patients (Maedica(Bucur), 2016, 11 (4): 320-324). Therefore, there is a great need for effective treatments of heart failure including HFpEF, HFrEF, and HFmrEF.

Mitochondria control metabolism in individual cells by burning sugars and fats. Mitochondrial uncoupling is a robust and natural process that the body utilizes to generate heat. Heat is generated by the mitochondrion via the uncoupling of respiration (Complexes I-IV) from ATP phosphorylation (Complex V). In fact, 20-40% of the calories consumed go toward the generation of body heat. Mitochondria-related disorders or conditions occur when mitochondria fail to produce enough energy for the body to function properly, affecting almost any part of the body including the cells of the brain, adipose tissue, nerves, muscles, heart, lungs, liver, kidneys, pancreas, eyes, and ears.

The administration of chemical uncouplers of mitochondria as a means to decrease fat deposits has been a scientific goal for many years. While there are several small molecules which uncouple mitochondrial oxidative phosphorylation, the most well-known is 2,4-dinitrophenol (DNP). Though DNP is known to uncouple with robust effect, it unfortunately is associated with an unacceptable high rate of significant adverse effects (J. Med. Toxicol. 2011 September; 7 (3): 205-212). These adverse effects may include hyperthermia, tachycardia, diaphoresis and tachypnoea, eventually leading to death. Being a small, highly permeable, lipophilic acid, DNP is rapidly absorbed in the stomach. The high concentration rapidly distributes and uncouples immediately, producing high levels of heat in a short period of time. Thus, DNP has a small therapeutic index and is extremely dangerous in overdose. DNP was labelled as “extremely dangerous and not fit for human consumption” by the Federal Food, Drug and Cosmetic Act of 1938. Accordingly, there is a need for uncouplers that can safely treat mitochondria-related disorders or conditions.

5-[(2,4-dinitrophenoxy)methyl]-1-methyl-2-nitro-1H-imidazole is a novel small molecule uncoupler (Compound 1). It works as a controlled metabolic accelerator (CMA). It is designed to effectively address the root cause of metabolic diseases, the accumulation of fat and sugars in the body. CMAs work to improve cellular metabolism and increase energy expenditure and calorie consumption, reducing the accumulation of fat. Using a new controlled and targeted approach, Compound 1 can increase mitochondrial proton leak, an ongoing process in the body that dissipates energy, and accounts for 20%-40% of daily calories. Compound 1 leverages a mitochondrial uncoupling mechanism to increase substrate utilization.

Compound 1 has been studied with nonclinical models. The demonstration of potent therapeutic activity in relevant rodent models of disease, together with the pharmacokinetics and safety profile, support that Compound I can be used beneficially and safely for treating a wide range of mitochondria-related diseases. It was also discovered that Compound 1 may be efficacious in treating cardiovascular diseases.

### SUMMARY

In some embodiments, the present disclosure provides a method for treating a cardiovascular disease in a subject comprising administering to the subject a therapeutically effective amount of Compound 1 or a pharmaceutically acceptable salt thereof.

In some embodiments, the present disclosure provides a method of treating heart failure, comprising administering to the subject a therapeutically effective amount of Compound 1 or a pharmaceutically acceptable salt thereof.

In some embodiments, the present disclosure provides a method of reducing a cardiovascular risk or mortality in a subject suffering from a symptom due to a cardiovascular disease, comprising administering to the subject a therapeutically effective amount of Compound 1 or a pharmaceutically acceptable salt thereof.

In some embodiments, the cardiovascular disease is selected from the group consisting of heart failure, heart attack, coronary artery disease, and coronary heart disease (CHD).

Heart failure as used herein includes heart failure with preserved ejection fraction (HFpEF), or heart failure with reduced ejection fraction (HFrEF), or heart failure with mid-range ejection fraction (HFmrEF).

In some embodiments, the cardiovascular disease is HFpEF.

In some embodiments, the cardiovascular disease is HFrEF.

In some embodiments, the cardiovascular disease is HFmrEF.

In some embodiments, the present disclosure provides a method for treating heart failure with preserved ejection fraction (HFpEF) in a subject, comprising administering to the subject a therapeutically effective amount of 5-[(2,4-dinitrophenoxy)methyl]-1-methyl-2-nitro-1H-imidazole, or a pharmaceutically acceptable salt thereof.

In some embodiments, the present disclosure provides a method for treating heart failure with reduced ejection fraction (HFrEF) in a subject, comprising administering to the subject a therapeutically effective amount of 5-[(2,4-dinitrophenoxy)methyl]-1-methyl-2-nitro-1H-imidazole, or a pharmaceutically acceptable salt thereof.

In some embodiments, the present disclosure provides a method for treating heart failure with mid-range ejection fraction (HFmrEF) in a subject, comprising administering to the subject a therapeutically effective amount of 5-[(2,4-dinitrophenoxy)methyl]-1-methyl-2-nitro-1H-imidazole or a pharmaceutically acceptable salt thereof.

In some embodiments, the subject is suffering from at least one of shortness of breath, shortness of breath with exertion, impaired energetics in the heart, dizziness, fatigue, dyspnea, palpitations (atrial fibrillation), chest discomfort, edema, syncope, and a limit on an activity of daily living.

In some embodiments, the limit on an activity of daily living is difficulties on personal care, mobility, and eating.

In some embodiments, the subject is suffering from symptoms selected from reduced exercise tolerance, fatigue, tiredness, increased time to recover after exercise, and ankle swelling.

In some embodiments, the subject is suffering from at least one of coronary artery disease, hypertension, and heart murmur.

In some embodiments, the present disclosure provides a method of reducing blood pressure in a subject comprising administering to the subject a therapeutically effective amount of Compound 1, or a pharmaceutically acceptable salt thereof.

In some embodiments, the subject is suffering from at least one of cardiovascular disease, hypertension, resistant hypertension, and severe hypertension.

In some embodiments, the cardiovascular disease is heart failure (which may be HFpEF, HFrEF, or HFmrEF), heart attack, coronary artery disease, or coronary heart disease (CHD). In some embodiments, the subject has hypertension associated with HFpEF.

In some embodiments, the subject has hypertension associated with HFrEF.

In some embodiments, the subject has hypertension associated with HFmrEF.

In some embodiments, wherein the subject experiences a reduction of blood pressure of at least 5 mmHg after the administration.

In some embodiments, the method reduces the risk of developing a cardiovascular disease, and/or reduces the risk of HFpEF, HFrEF, or HFmrEF.

In some embodiments, the method slows the progression of HFpEF, HFrEF, or HFmrEF.

In some embodiments, the method comprises at least one of:
i) extending the half-life (t_{1/2}) of DNP;
ii) delaying the time to maximum plasma concentration (Tₘₐₓ) of DNP;
iii) lowering maximum plasma concentration (Cₘₐₓ) of DNP; and
iv) increasing area under the curve (AUC).

In some embodiments, the subject does not experience significant systemic toxicity, side effects, significant increase in body temperature, or significant increase in heart rate after administration.

In some embodiments, the present disclosure provides a method of treating a cardiovascular disease by achieving:
i) asteady state of maximum plasma concentration (Cₘₐₓ) of DNP from about 80 ng/ml to about 8300 ng/ml;
ii) mean half-life (t_{1/2}) of DNP about 20-50 hours, about 25-40 hours, or about 30-40 hours;
iii) median time to maximum plasma concentration (Tₘₐₓ) of DNP about 6-8 hours or about 6-10 hours;
iv) median area under the curve extrapolated to infinity (AUC_{inf}) of DNP about 3 h*μg/mL to about 420 h*μg/mL; and
AUC/Cₘₐₓ ratio of about 18, by administering to a subject about 30 mg to about 1400 mg of Compound 1 or a pharmaceutically acceptable salt thereof.

In some embodiments, the present disclosure provides a method of treating mitochondria-related disorders or conditions without causing a clinically significant risk of adverse events in a subject, the method comprising administering to the subject a therapeutically effective amount of Compound 1 or a pharmaceutically acceptable salt thereof.

In some embodiments, the present disclosure provides a method of reducing toxicity or side effects in treating mitochondria-related disorders or conditions in a subject, comprising administering to the subject a therapeutically effective amount of Compound 1 or a pharmaceutically acceptable salt thereof.

In some embodiments, the present disclosure provides a method of preventing overdose in treating mitochondria-related disorders or conditions in a subject, comprising administering to the subject a therapeutically effective amount of Compound 1 or a pharmaceutically acceptable salt thereof.

In some embodiments, the present disclosure provides a method for increasing metabolic rate or resting energy expenditure without causing a clinically significant risk of adverse events in a subject, comprising administering to the subject a therapeutically effective amount of Compound 1 or a pharmaceutically acceptable salt thereof.

In some embodiments, the present disclosure provides a method for treating dysmetabolism in a subject comprising administering to the subject a therapeutically effective amount of Compound 1, or a pharmaceutically acceptable salt thereof.

In some embodiments, the present disclosure provides a method of treating hypertriglyceridemia associated with cardiovascular disease, atherosclerosis, obesity, hypertension, diabetes, insulin resistance, and/or liver disease in a subject, comprising administering to the subject a therapeutically effective amount of Compound 1, or a pharmaceutically acceptable salt thereof.

In some embodiments, the present disclosure provides a method of treating severe hypertriglyceridemia in a subject, comprising administering to the subject a therapeutically effective amount of Compound 1, or a pharmaceutically acceptable salt thereof.

In some embodiments, the present disclosure provides a method of reducing liver fat by at least 50% or a method of reducing lipids by at least 10% in a subject, the method comprising administering to the subject a therapeutically effective amount of Compound 1, or a pharmaceutically acceptable salt thereof.

In some embodiments, the present disclosure provides a method of treating obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, or hepatocellular carcinoma, the method comprising administering a therapeutically effective amount of Compound 1 or a pharmaceutically acceptable salt thereof in a subject, to achieve at least one of:
i) a steady state of maximum plasma concentration (Cₘₐₓ) of 2,4-dinitrophenol from about 80 ng/ml to about 8300 ng/ml;
ii) mean half-life (t_{1/2}) of 2,4-dinitrophenol about 20-50 hours, about 25-40 hours, or about 30-40 hours;
iii) median time to maximum plasma concentration (Tₘₐₓ) of 2,4-dinitrophenol about 6-8 hours or about 6-10 hours;
iv) median area under the curve extrapolated to infinity (AUC_{inf}) of 2,4-dinitrophenol about 3 h*μg/mL to about 420 h*μg/mL; and
v) AUC/Cₘₐₓ ratio of about 18.

### BRIEF DESCRIPTION OF THE DRAWINGS

FIG. 1 illustrates the plasma concentration of 2,4-dinitrophenol after administration of Compound 1 and 2,4-dinitrophenol in dog.

FIGS. 2A and 2B illustrate the AUC of 2,4-dinitrophenol after Compound 1 administration.

FIGS. 3A and 3B illustrate the plasma Compound 1 concentration by food status following 500 mg Compound 1 oral administration (Linear and Semi-log Scale).

FIGS. 4A and 4B illustrate the plasma 2,4-dinitrophenol concentration by food status following 500 mg Compound 1 oral administration (Linear and Semi-log Scale).

FIG. 5 illustrates the 2,4-dinitrophenol curve for all cohorts.

FIG. 6 illustrates the body temperature of MAD cohorts during MAD QD dosing period.

FIG. 7 illustrates the Resting Energy Expenditure (REE) MAD cohorts during MAD QD dosing period.

FIG. 8 illustrates body weight reduction after administering Compound 1

FIG. 9 illustrates average glucose level reduction after administering Compound 1.

FIG. 10 illustrates the change in Systolic Blood Pressure after administering Compound 1.

FIG. 11 illustrates the change in Diastolic Blood Pressure after administering Compound 1.

FIG. 12 illustrates the observed heart rate after administering of Compound 1.

FIG. 13 illustrates the observed body temperature changes after administering of Compound 1.

FIG. 14 illustrates the observed PK result after administering of Compound 1.

FIG. 15 illustrates the placebo-Corrected percent change from baseline values for MRI-proton Density fat fraction (PDFF) after administering of Compound 1.

FIG. 16 illustrates an analysis of Mean change from baseline at Day 61 of FAS population of the covariance for MRI-proton density fat fraction (PDFF).

FIG. 17 illustrates the mean change from baseline FAS population in a repeated measure analysis for InBody body weight.

FIG. 18 illustrates the change in observed body weight after administering of Compound 1

FIG. 19 illustrates the placebo-corrected percent change from baseline values for abdominal MRI liver volume and adiposity by treatment group FAS population.

FIG. 20 illustrates the placebo-corrected percent change from baseline values for abdominal MRI liver volume and adiposity by treatment group FAS population.

FIG. 21 illustrates the fat loss (total adipose tissue) confirmed by MRI.

FIG. 22 illustrates the observed changed in liver volume.

FIG. 23 illustrates the observed mean change from baseline at Day 61 in systolic blood pressure

FIG. 24 illustrates the observed mean change from baseline at Day 61 in diastolic blood pressure.

FIG. 25 illustrates the observed mean change from baseline at Day 61 in High Sensitivity C-reactive protein (hsCRP).

### DETAILED DESCRIPTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

5-[(2,4-Dinitrophenoxy)methyl]-1-methyl-2-nitro-1H-imidazole is a novel small molecule uncoupler. It has the following structure:

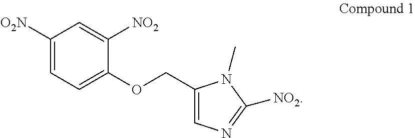 [SMILES: Cn1c(COc2ccc([N+](=O)[O-])cc2[N+](=O)[O-])cnc1[N+](=O)[O-]]

5-[(2,4-Dinitrophenoxy)methyl]-1-methyl-2-nitro-1H-imidazole may be prepared by the procedures described in WO 2018/129258.

In this disclosure, Compound 1 and CM1 are interchangeable. They both refer to 5-[(2,4-Dinitrophenoxy)methyl]-1-methyl-2-nitro-1H-imidazole.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY, 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, “about” means a range extending to +/−10%, +/−5%, or +/−2% of the specified value. In some embodiments, “about” means the specified value.

As used herein, “treatment” or “treating” or “palliating” or “ameliorating” or “reducing” are used interchangeably herein. These terms refer to an approach for obtaining beneficial or desired results including but not limited to a therapeutic benefit. By therapeutic benefit means eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. Treatment includes causing the clinical symptoms of the disease to slow in development by administration of a composition; suppressing the disease, that is, causing a reduction in the clinical symptoms of the disease; inhibiting the disease, that is, arresting the development of clinical symptoms by administration of a composition after the initial appearance of symptoms; and/or relieving the disease, that is, causing the regression of clinical symptoms by administration of a composition after their initial appearance.

“Patient” or “subject” or “subject in need thereof” refers to a living organism suffering from or prone to a disease or condition that can be treated by using the methods provided herein. The term does not necessarily indicate that the subject has been diagnosed with a particular disease, but typically refers to an individual under medical supervision. Non-limiting examples include humans, other mammals, bovines, rats, mice, dogs, cats, monkeys, goat, sheep, cows, deer, and other non-mammalian animals. In some embodiments, a patient, subject or subject in need thereof is a human.

As used herein, “administration” of a disclosed compound encompasses the delivery to a subject of a compound as described herein, or a prodrug or other pharmaceutically acceptable derivative thereof, using any suitable formulation or route of administration, e.g., as described herein.

“Pharmaceutically acceptable” refers to compounds, salts, compositions, dosage forms and other materials that are useful in preparing a pharmaceutical composition that is suitable for veterinary or human pharmaceutical use.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). Efficacy can also be expressed as “-fold” increase or decrease. For example, a therapeutically effective amount can have at least a 1.2-fold, 1.5-fold, 2-fold, 5-fold, or more effect over a control.

As used herein, the term “significant increase in body temperature” in a subject refers to a body temperature increase that is associated with deleterious effects on the subject, not limited to illness, physical discomfort or pain, coma and death. In one non-limiting embodiment, the significant increase in body temperature is an increase of about 0.5° C., about 1° C., about 1.5° C., about 2° C., about 2.5° C., about 3° C., about 3.5° C., about 4° C., about 4.5° C., about 5° C., about 5.5° C., about 6° C. or higher. In another non-limiting embodiment, the significant increase in body temperature lasts for about 5 min, about 15 min, about 30 min, about 45 min, about 1 h, about 1.5 h, about 2 h, about 3 h, about 4 h, about 5 h, about 6 h, about 7 h, about 8 h, about 9 h, about 10 h, about 12 h, about 14 h, about 16 h, about 18 h, about 20 h, about 22 h, about 24 h or longer.

As used herein, the term “significant systemic toxicity” in a subject refers to systemic toxicity that is associated with deleterious effects on the subject, not limited to illness, physical discomfort or pain, coma and death. In one non-limiting embodiment, significant systemic toxicity is indicated by increase in levels of liver enzymes, blood urea nitrogen or creatinine as compared to the corresponding levels in the subject in the absence of administration of the composition.

### Methods of Treatment

In some embodiments, the present disclosure provides a method for treating a cardiovascular disease in a subject comprising administering to the subject a therapeutically effective amount of Compound 1 or a pharmaceutically acceptable salt thereof. Initial studies suggest that Compound 1 can achieve the following:
Controlled and improved 2,4-dinitrophenol PK-low Cₘₐₓ, high AUC, low variability.
Potential once daily oral administration.
Liver specific uncoupling and reduced systemic effects.
A wide therapeutic index.
   Thus, Compound 1 is useful in treating a wide range of diseases safely.

In some embodiments, the present disclosure provides a method of treating heart failure in a subject suffering from a symptom due to a cardiovascular disease, comprising administering to the subject a therapeutically effective amount of Compound 1 or a pharmaceutically acceptable salt thereof.

In some embodiments, the present disclosure provides a method of reducing a cardiovascular risk or mortality in a subject suffering from a symptom due to a cardiovascular disease, comprising administering to the subject a therapeutically effective amount of Compound 1 or a pharmaceutically acceptable salt thereof.

In some embodiments, the symptom due to a cardiovascular disease is shortness of breath, dizziness, chest pain, syncope, fatigue, or limits on activities of daily living.

In some embodiments, the limit on an activity of daily living is difficulties on personal care, mobility, or eating.

In some embodiments, the present disclosure provides a method of treating cardiovascular diseases in a subject, comprising administering to the subject a therapeutically effective amount of Compound 1 or a pharmaceutically acceptable salt thereof.

In some embodiments, the cardiovascular diseases are associated with obesity. In some embodiments, the cardiovascular diseases include the following diseases, disorders, or conditions.

Disrupted cardiovascular hemodynamics characterized by increased heart rate among those with physical inactivity, increased risk for atrial fibrillation, increased blood volume, increased cardiac output, increased systemic vascular resistance among those with hypertension and insulin resistance, increased arterial pressure, increased left ventricular wall stress, increased pulmonary artery pressure, alternations in ventricular pressure among those with sleep apnea.

Atherosclerosis and myocardial infarction which may increase indirectly through promotion of major atherosclerotic risk factors (e.g., diabetes mellitus, hypertension, dyslipidemia) or directly through adiposopathic endocrinopathies and immunopathies of epicardial adipose tissue.

Epicardial fat accumulation, pathogenic paracrine and vasocrine signaling, increased inflammatory macrophages, increased T-Lymphocytes and mast cells, increased adiposopathic adipokines, and reduced vasculoprotective adipokines

Heart failure (HF), especially HF with preserved ejection fraction (HFpEF)

Atherosclerotic Cardiovascular Disease (ASCVD), dysrhythmias, fatty infiltration of the heart, increased coronary calcium.

Sleep apnea that may lead to hypoxia, increased epinephrine (adrenaline) may lead to high blood pressure, swings in thoracic pressures increase left and right heart ventricular pressure.

Thrombosis and thromboembolic events, increased adipose tissue compresses pelvic and lower extremities veins, impairs venous return, and promotes deep vein thrombosis.

Abnormal heart cell and structure characterized by myocardial steatosis, apoptosis and fibrosis as well as left ventricular remodeling and hypertrophy, left atrial enlargement, right ventricular hypertrophy and increased pericardial and perivascular adipose tissue.

Reduced heart function characterized by hypoxia due to sleep apnea, atherosclerosis, thrombosis, left ventricular dysfunction (both diastolic and systolic) and right ventricular failure.

Immunopathies characterized by increased pro-inflammatory adipocytokines e.g. tumor necrosis factor (TNF), interleukins such as interleukin-6 (IL-6), monocyte chemoattractant protein-1 (MCP-1) or C-reactive protein (CRP) or decreased anti-inflammatory adipocytokines (e.g., adiponectin) and IL-10.

Immonopathies characterized by increased neutrophilic activation and granulation such as severe asthma, and glucocorticoid resistant severe asthma.

Endocrinopathies characterized by activation of the renin-angiotensin-aldosterone system leading to elevated blood pressure, alteration of peroxisome proliferator activated receptor expression

Endocrinopathies characterized by hyperinsulinemia, systemic insulin resistance and adiposopathy, myocardial insulin insensitivity.

Endocrinopathies characterized by leptin insensitivity, with increased leptin levels potentially contributing to cardiac hypertrophy and heart failure.

Lipotoxicity characterized by limitations of energy storage in peripheral subcutaneous adipose tissue.

Overflow of free fatty acid delivery to liver, muscle, pancreas, kidney and/or visceral, pericardial, and perivascular adipose tissue

In some embodiments, the cardiovascular disease is heart failure, heart attack, coronary artery disease, and coronary heart disease (CHD).

In some embodiments, heart failure includes HFpEF, HFrEF, or HFmrEF.

In some embodiments, the subject experiences a reduction in the risk of a major cardiovascular event after administration.

In some embodiments, the major cardiovascular event is death or hospitalization for worsening of the disease.

In some embodiments, the present disclosure provides a method for treating heart failure with preserved ejection fraction (HFpEF) in a subject, comprising administering to the subject a therapeutically effective amount of Compound 1 or a pharmaceutically acceptable salt thereof.

In some embodiments, the present disclosure provides a method for treating heart failure with reduced ejection fraction (HFrEF) in a subject, comprising administering to the subject a therapeutically effective amount of Compound 1 or a pharmaceutically acceptable salt thereof.

In some embodiments, the present disclosure provides a method for treating heart failure with mid-range ejection fraction (HFmrEF) in a subject, comprising administering to the subject a therapeutically effective amount of Compound 1 or a pharmaceutically acceptable salt thereof.

In some embodiments, the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

In some embodiments, the subject is suffering from at least one symptoms selected from shortness of breath, shortness of breath with exertion, impaired energetics in the heart, dizziness, fatigue, dyspnea, palpitations (atrial fibrillation), chest discomfort, edema, syncope, and a limit on an activity of daily living.

In some embodiments, the limit on an activity of daily living is difficulties on personal care, mobility, and eating.

In some embodiments, the subject is suffering from at least one of reduced exercise tolerance, fatigue, tiredness, increased time to recover after exercise, and ankle swelling.

In some embodiments, the subject is suffering from at least one of coronary artery disease, hypertension, and heart murmur.

In some embodiments, wherein the subject experiences an improvement of cardiac bioenergetic deficiency after administration, wherein the improvement comprises weight loss>5%, reduction in blood pressure, increased quality of life, increased exercise tolerance, and/or a reduction in the risk of a major cardiovascular event, wherein the major cardiovascular event is selected from the group consisting of death, hospitalization for worsening of the disease, and myocardial infraction.

In some embodiments, the method further comprises assessing peak oxygen consumption (VO₂) and/or VE/CO₂ or VE/VCO₂ slope in the subject during exercise before and after administration of the therapeutically effective amount of Compound 1, wherein an increase in VO₂ in the subject after administration indicates a reduction in the extent of HFpEF, HFrEF, HFmrEF, or one or more symptomatic component or condition of cardiovascular diseases thereof in the subject.

In some embodiments, the method increases VO₂ in the subject after administration. In some embodiments, the method increases the subjects exercise tolerance.

In some embodiments, the method increases the subjects exercise tolerance as measured by assessing 6-minute walk distance (6MWD) before and after administration of the therapeutically effective amount of Compound 1, wherein an increase in 6MWD in the subject after administration indicates a reduction in the extent of HFpEF or the at least one symptomatic component or condition thereof in the subject.

In some embodiments, the method increases 6MWD after the administration.

In some embodiments, the HFpEF in the subject is diagnosed according to echocardiography (E/e′) or biomarkers (NT-proBNP).

In some embodiments, the method further comprises assessing a NYHA classification score of the subject before and after administration.

The NYHA functional classification grades the severity of heart failure symptoms as one of four functional classes. The NYHA functional classification is widely used in clinical practice and in research because it provides a standard description of severity that can be used to assess response to treatment and to guide management. The NYHA functional classification based on severity of symptoms and physical activity are:
Class I: No limitation of physical activity. Ordinary physical activity does not cause undue breathlessness, fatigue, or palpitations
Class II: Slight limitation of physical activity. Comfortable at rest, but ordinary physical activity results in undue breathlessness, fatigue, or palpitations.
Class III: Marked limitation of physical activity. Comfortable at rest, but less than ordinary physical activity results in undue breathlessness, fatigue, or palpitations.
Class IV: Unable to carry on any physical activity without discomfort. Symptoms at rest can be present. If any physical activity is undertaken, discomfort is increased.

In some embodiments, the method further comprises the step of: assessing a NYHA classification score of the subject before and after administration of the therapeutically effective amount of Compound 1, wherein a decreased NYHA score after administration indicates a reduction in the extent of the disease in the subject.

In some embodiments, the method decreases the NYHA classification score of the subject after administration from Class III to Class II, or from Class II to Class I.

In some embodiments, the method increases the subject's quality of life.

In some embodiments, the method increases the subjects quality of life as assessed by a standardized questionnaire such as KCCQ (Kansas City Cardiomyopathy Questionnaire), KCCQ-12, KCCQ-Physical Limitation Score (KCCQ-PLS), KCCQ-Totally Symptom Score (KCCQ-TSS) KCCQ-Clinical Summary Score (KCCQ-CSS), KCCQ-Overall Summary Score (KCCQ-OSS) or other derivatives.

In some embodiments, the present disclosure provides a method of reducing blood pressure in a subject comprising administering to the subject a therapeutically effective amount of Compound 1, or a pharmaceutically acceptable salt thereof.

In some embodiments, the subject is suffering from at least one of: cardiovascular disease, hypertension, resistant hypertension, and severe hypertension.

In some embodiments, the cardiovascular disease is selected from the group consisting of heart failure, HFpEF, HFrEF, heart attack, coronary artery disease, and coronary heart disease (CHD).

In some embodiments, the subject has hypertension associated with HFpEF.

In some embodiments, the subject has hypertension associated with HFrEF.

In some embodiments, the subject has hypertension associated with HFmrEF.

In some embodiments, the subject is suffering from at least one of symptoms selected from headaches, shortness of breath, chest pain, nosebleeds, dizziness, fatigue, vision problem, irregular heartbeat, blood in urine, sweating, trouble sleeping, and blood spots in eyes.

In some embodiments, the symptoms are associated with HFpEF, HFrEF, or HFmrEF.

In some embodiments, wherein the reducing blood pressure comprises reducing diastolic blood pressure and/or reducing systolic blood pressure.

In some embodiments, wherein the subject experiences a reduction of blood pressure of at least 5 mmHg after the administration.

In some embodiments, the method reduces the risk of developing a cardiovascular disease, reduces the risk of HFpEF, or slows the progression of HFpEF.

In some embodiments, the method reduces the risk of developing a cardiovascular disease, reduces the risk of HFrEF, or slows the progression of HFrEF.

In some embodiments, the method reduces the risk of developing a cardiovascular disease, reduces the risk of HFmrEF, or slows the progression of HFmrEF.

In some embodiments, the subject is in a fasted condition before administration. In some embodiments, the subject is in a fed condition before administration.

In some embodiments, the subject experiences a reduction in at least one of body weight, blood pressure, and blood glucose after the administration.

In some embodiments, the subject experiences at least one of:
i) a reduction of body weight by at least 5% or at least 10%;
ii) a reduction of blood pressure of at least 5 mmHg;
iii) a reduction of HbA_{1c} by at least 0.5%, or at least 1.5%;
iv) a reduction of lipids by at least 10%; and
v) a reduction of liver fat by at least 50%.

In some embodiments, the method comprises at least one of:
extending the half-life (t_{1/2}) of 2,4-dinitrophenol;
delaying the time to maximum plasma concentration (Tₘₐₓ) of 2,4-dinitrophenol;
lowering maximum plasma concentration (Cₘₐₓ) of 2,4-dinitrophenol; and
increasing area under the curve (AUC).

In some embodiments, the mean half-life of 2,4-dinitrophenol is extended to about 20-50 hours, 25-40 hours, or 30-40 hours.

In some embodiments, the median Tₘₐₓ of 2,4-dinitrophenol is extended to at least 6 hours or at least 8 hours.

In some embodiments, the median Tₘₐₓ of 2,4-dinitrophenol is extended to about 6-8 hours or about 6-10 hours.

In some embodiments, lowering 2,4-dinitrophenol Cₘₐₓ comprises providing a steady state of Cₘₐₓ of 2,4-dinitrophenol from about 80 ng/mL to about 8300 ng/mL in the subject after administration.

In some embodiments, the method provides an AUC/Cₘₐₓ ratio of about 18 in the subject.

In some embodiments, the subject does not experience significant systemic toxicity, side effects, significant increase in body temperature, or significant increase in heart rate after administration.

In some embodiments, the side effects comprise at least one of nausea, vomiting, sweating, dizziness, headaches, cataracts, glaucoma, pyrexia, hyperthermia, tachycardia, diaphoresis, tachypnoea, and death.

In some embodiments, the present disclosure provides a method of treating a cardiovascular disease, the method comprising administering to a subject about 30 mg to about 1400 mg of Compound 1 or a pharmaceutically acceptable salt thereof to achieve at least one of:
i) a steady state of maximum plasma concentration (Cₘₐₓ) of 2,4-dinitrophenol from about 80 ng/ml to about 8300 ng/ml;
ii) mean half-life (t_{1/2}) of 2,4-dinitrophenol about 20-50 hours, about 25-40 hours, or about 30-40 hours;
iii) median time to maximum plasma concentration (Tₘₐₓ) of 2,4-dinitrophenol about 6-8 hours or about 6-10 hours;
iv) median area under the curve extrapolated to infinity (AUC_{inf}) of 2,4-dinitrophenol about 3 h*μg/mL to about 420 h*μg/mL; and
v) AUC/Cₘₐₓ ratio of about 18.

In some embodiments, the present disclosure provides a method of treating mitochondria-related disorders or conditions without causing a clinically significant risk of adverse events in a subject, the method comprising administering to the subject a therapeutically effective amount of Compound 1 or a pharmaceutically acceptable salt thereof.

In some embodiments, the present disclosure provides a method of reducing toxicity or side effects in treating mitochondria-related disorders or conditions in a subject, comprising administering to the subject a therapeutically effective amount of Compound 1 or a pharmaceutically acceptable salt thereof.

In some embodiments, the present disclosure provides a method of reducing toxicity or side effects of 2,4-dinitrophenol in treating mitochondria-related disorders or conditions in a subject, comprising administering to the subject a therapeutically effective amount of Compound 1 or a pharmaceutically acceptable salt thereof.

In some embodiments, the present disclosure provides a method of preventing overdose in treating mitochondria-related disorders or conditions in a subject, comprising administering to the subject a therapeutically effective amount of Compound 1 or a pharmaceutically acceptable salt thereof.

In some embodiments, the present disclosure provides a method of preventing overdose of 2,4-dinitrophenol in treating mitochondria-related disorders or conditions in a subject, comprising administering to the subject a therapeutically effective amount of Compound 1 or a pharmaceutically acceptable salt thereof.

In some embodiments, the mitochondria-related disorder comprises obesity, excess body fat, diabetes, insulin resistance or intolerance, high blood pressure, dyslipidemia, cardiovascular disease, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, or liver disease.

In some embodiments, the disorder are Branched Chain Amino Acid (BCAA) metabolism disorders, lysosomal storage disorders, glycogen storage disorders. In some embodiments, the diabetes is type 2 diabetes (T2DM).

In some embodiments, the cardiovascular disease comprises heart failure, HFpEF, HFrEF, HFmrEF, heart attack, coronary artery disease, or CHD.

In some embodiments, the liver disease comprises non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

In some embodiments, the mitochondria-related disorder comprises cardiovascular disease, hypertension, type 2 diabetes, dyslipidemia, obesity, or non-alcoholic steatohepatitis (NASH).

In some embodiments, the mitochondria-related condition is at least on of steatosis, inflammation, fibrosis, cirrhosis, and hepatocyte injury in NASH.

In some embodiments, the toxicity, adverse events, side effects, and overdose are associated with a mitochondria uncoupler.

In some embodiments, the mitochondria uncoupler is 2,4-dinitrophenol.

In some embodiments, the method comprises at least one of:
i) extending the half-life (t_{1/2}) of 2,4-dinitrophenol;
ii) delaying the time to maximum plasma concentration (Tₘₐₓ) of 2,4-dinitrophenol;
iii) lowering maximum plasma concentration (Cₘₐₓ) of 2,4-dinitrophenol; and
iv) increasing the area under the curve (AUC).

In some embodiments, the present disclosure provides a method for increasing metabolic rate without causing a clinically significant risk of adverse events in a subject, comprising administering to the subject a therapeutically effective amount of Compound 1 or a pharmaceutically acceptable salt thereof.

In some embodiments, the present disclosure provides a method for increase resting energy expenditure in a subject comprising administering to the subject a therapeutically effective amount of Compound 1 or a pharmaceutically acceptable salt thereof.

In some embodiments, the present disclosure provides a method for treating dysmetabolism in a subject comprising administering to the subject a therapeutically effective amount of Compound 1 or a pharmaceutically acceptable salt thereof.

In some embodiments, the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

In some embodiments, the method comprises increasing resting metabolic rate without causing a clinically significant risk of adverse events.

In some embodiments, the resting metabolic rate is increased by at least 10%.

In some embodiments, the resting metabolic rate is increased by at least 20%.

In some embodiments, the subject experiences an increase of resting energy expenditure of at least 10% after the administration.

In some embodiments, the subject experiences an increase of resting energy expenditure of at least 20% after the administration.

In some embodiments, the subject experiences an increase of resting energy expenditure of about 30% after the administration.

In some embodiments, the method slows the progression of at least one of atherosclerosis, NAFLD, NASH, noncirrhotic NASH, noncirrhotic NASH with liver fibrosis, hepatic steatosis, hepatic fibrosis, liver cirrhosis, and hepatocellular carcinoma.

In some embodiments, the method accelerates human body's natural processes to improve cardio-metabolic processes.

In some embodiments, the present disclosure provides a method of treating hypertriglyceridemia associated with cardiovascular disease, atherosclerosis, obesity, hypertension, diabetes, insulin resistance, and/or liver disease in a subject, comprising administering to the subject a therapeutically effective amount of Compound 1 or a pharmaceutically acceptable salt thereof.

In some embodiments, the subject has moderate hypertriglyceridemia associated with cardiovascular disease, atherosclerosis, obesity, hypertension, diabetes, insulin resistance, and/or liver disease; or severe hypertriglyceridemia associated with cardiovascular disease, atherosclerosis, obesity, hypertension, diabetes, insulin resistance, and/or liver disease.

In some embodiments, the present disclosure provides a method of treating severe hypertriglyceridemia in a subject, comprising administering to the subject a therapeutically effective amount of Compound 1 or a pharmaceutically acceptable salt thereof.

In some embodiments, the subject has a triglyceride blood level above 500 mg/dL.

In some embodiments, the subject has severe hypertriglyceridemia associated with cardiovascular disease, atherosclerosis, obesity, hypertension, diabetes, insulin resistance, and/or liver disease.

In some embodiments, the subject has treatment resistant hypertriglyceridemia.

In some embodiments, the subject has treatment resistant severe hypertriglyceridemia.

In some embodiments, the subject has treatment resistant severe hypertriglyceridemia associated with cardiovascular disease, atherosclerosis, obesity, hypertension, diabetes, insulin resistance, and/or liver disease.

In some embodiments, the subject is suffering from at least one of abdominal pain, pain in the mid-epigastric, chest, or back regions, gastrointestinal pain, difficulty breathing, loss of appetite, nausea, vomiting, inflammation of the pancreas, memory loss, dementia, xanthelasmas, corneal arcus, and xanthomas.

In some embodiments, the subject is an adult male subject.

In some embodiments, the subject is a Hispanic descendant.

In some embodiments, the method comprises lowering low-density lipoprotein cholesterol levels and/or lowering non-high-density lipoprotein cholesterol levels.

In some embodiments, the method comprises at least one of:
i) lowering triglyceride levels by at least 5%, at least 10%, or at least 20%;
ii) lowering low-density lipoprotein cholesterol levels by at least 5%, at least 10%, or at least 20%; and
iii) lowering non-high-density lipoprotein cholesterol levels by at least 5%, at least 10%, or at least 20%.

In some embodiments, the present disclosure provides a method of reducing liver fat by at least 50% in a subject, comprising administering to the subject a therapeutically effective amount of Compound 1 or a pharmaceutically acceptable salt thereof.

In some embodiments, the present disclosure provides a method of reducing lipids by at least 10% in a subject, comprising administering to the subject a therapeutically effective amount of Compound 1 or a pharmaceutically acceptable salt thereof.

In some embodiments, the present disclosure provides a method of treating or reducing the risk of cancer in a subject, comprising administering to the subject a therapeutically effective amount of Compound 1 or a pharmaceutically acceptable salt thereof.

In some embodiments, the cancer includes biliary tract cancer, bladder cancer, brain cancer (i.e., meningiomas), breast cancer (postmenopausal), cervical cancer, colorectal cancer, endometrial/uterine cancer, esophageal cancer, gallbladder cancer, head and neck cancer, kidney/renal cancer, leukemia, liver cancer, multiple myeloma, non-Hodgkin lymphoma, ovarian cancer, pancreatic cancer, stomach cancer and thyroid cancer, and prostate cancer.

In some embodiments, the cancer is associated with obesity, excess body fat, diabetes, high blood pressure, dyslipidemia, metabolic diseases, liver diseases, and/or cardiovascular diseases.

In some embodiments, the present disclosure provides a method of treating obesity, cancer, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, or hepatocellular carcinoma, the method comprising administering a therapeutically effective amount of Compound 1 or a pharmaceutically acceptable salt thereof in a subject, to achieve at least one of:
i) a steady state of maximum plasma concentration (Cₘₐₓ) of 2,4-dinitrophenol from about 80 ng/ml to about 8300 ng/ml;
ii) median half-life (t_{1/2}) of 2,4-dinitrophenol about 20-50 hours, about 25-40 hours, or about 30-40 hours;
iii) median time to maximum plasma concentration (Tₘₐₓ) of 2,4-dinitrophenol about 6-8 hours or about 6-10 hours;
iv) median area under the curve extrapolated to infinity (AUC_{inf}) of 2,4-dinitrophenol about 3 h*μg/mL to about 420 h*μg/mL; and
v) AUC/Cₘₐₓ ratio of about 18.

In some embodiments, the method further comprises the step of: determining Fibroscan′ Vibration-controlled Transient Elastography (VCTE), Fibroscan® Controlled Attenuation Parameter (CAP) score, Magnetic resonance imaging proton density fat fraction (MRI-PDFF), and Enhanced Liver Fibrosis (ELF) score of the subject before and after administration.

In some embodiments, the subject has CAP score of greater than 300 dB/m before administration.

In some embodiments, the subject has at least 8% liver fat by MRI-PDFF before administering.

In some embodiments, the subject has elevated Body Mass Index (BMI).

In some embodiments, the subject has BMI of about 28.0 kg/m² to about 45.0 kg/m².

In some embodiments, the diabetes is type 2 diabetes (T2DM).

In some embodiments, the cardiovascular disease comprises heart failure, HFpEF, HFrEF, HFmrEF, heart attack, coronary artery disease, or CHD.

In some embodiments, the liver disease comprises non-alcoholic fatty liver disease (NAFLD), non-alcoholic steatohepatitis (NASH), noncirrhotic NASH, noncirrhotic NASH with liver fibrosis, hepatic steatosis, hepatic fibrosis, liver cirrhosis, or hepatocellular carcinoma.

In some embodiments, the mitochondria-related condition is at least on of steatosis, inflammation, fibrosis, cirrhosis, and hepatocyte injury in NASH.

In some embodiments, the subject is suffering from non-alcoholic fatty liver disease (NAFLD), non-alcoholic steatohepatitis (NASH), and/or hepatic steatosis.

In some embodiments, the subject suffers from type 2 diabetes, obesity, HFpEF, HFrEF, NAFLD, and/or NASH.

In some embodiments, the subject suffers from inflammation, fibrosis, cirrhosis in liver.

In some embodiments, the subject does not experience significant systemic toxicity, serious side effects, a clinically significant risk of adverse events, and/or overdoes after administration.

In some embodiments, the toxicity, adverse events, and side effects are associated with a mitochondria uncoupler.

In some embodiments, the mitochondria uncoupler is 2,4-dinitrophenol.

In some embodiments, the subject does not experience a clinically significant risk of adverse events, side effects, toxicity, and/or overdoes associated with 2,4-dinitrophenol. Thus, the subject in need can be safely treated without the danger of serious side effects and overdose.

In some embodiments, the adverse events or side effects comprise at least one of nausea, vomiting, sweating, dizziness, headaches, cataracts, glaucoma, pyrexia, hyperthermia, tachycardia, diaphoresis, tachypnoea, and death.

In some embodiments, the adverse events or side effects comprise at least one of pyrexia, hyperthermia, tachycardia, diaphoresis, tachypnoea, and death.

In some embodiments, the adverse event or side effect is characterized by at least one of elevated body temperature, elevated heart rate, abnormal sweating, erythema, perspiration, dehydration, and abnormally rapid breathing.

In some embodiments, the adverse event or side effect is associated with cardiovascular collapse, cardiac arrest, and/or death.

In some embodiments, the adverse event or side effect is associated with cardiac arrest.

In some embodiments, the subject does not experience a significant increase in body temperature or a significant increase in heart rate.

In some embodiments, the subject experiences a saturable absorption of Compound 1 such that overdose is prevented. In some embodiments, there is a saturation of absorption at high single doses. In some embodiments, there is a saturation of absorption at a single oral dose of above 500 mg, above 600 mg, above 700 mg, above 800 mg, above 900 mg, above 1000 mg, above 1050 mg, above 1100 mg, above 1200 mg, above 1300 mg, or above 1400 mg of Compound 1.

In some embodiments, the subject does not experience a correlation between dose and toxicity, adverse events, side effects, or overdose.

In some embodiments, the clinically significant risk of adverse events, side effects, toxicity, and/or overdoes is prevented by at least one of:
i) extending the half-life (t_{1/2}) of 2,4-dinitrophenol;
ii) delaying the time to maximum plasma concentration (Tₘₐₓ) of 2,4-dinitrophenol;
iii) lowering maximum plasma concentration (Cₘₐₓ) of 2,4-dinitrophenol; and
iv) increasing the area under the curve (AUC).

In some embodiments, the clinically significant risk of adverse events, side effects, toxicity, and/or overdoes is prevented by providing a steady state of maximum plasma concentration (Cₘₐₓ) of 2,4-dinitrophenol from about 80 ng/mL to about 8300 ng/ml with administration of Compound 1.

In some embodiments, the clinically significant risk of adverse events, side effects, toxicity, and/or overdoes is prevented by providing a mean half-life (t_{1/2}) of 2,4-dinitrophenol about 20-50 hours, about 25-40 hours, or about 30-40 hours with administration of Compound 1.

In some embodiments, the clinically significant risk of adverse events, side effects, toxicity, and/or overdoes is prevented by providing a median time to maximum plasma concentration (Tₘₐₓ) of 2,4-dinitrophenol about 6-8 hours or about 6-10 hours with administration of Compound 1.

In some embodiments, the clinically significant risk of adverse events, side effects, toxicity, and/or overdoes is prevented by providing a median area under the curve extrapolated to infinity (AUC_{inf}) of 2,4-dinitrophenol about 3 h*μg/mL to about 420 h*μg/mL with administration of Compound 1.

In some embodiments, the clinically significant risk of adverse events, side effects, toxicity, and/or overdoes is prevented by providing an AUC/Cₘₐₓ ratio of about 18 with administration of Compound 1.

In some embodiments, the above various methods include providing at least one of

i) median time to maximum plasma concentration (Tₘₐₓ) of Compound 1 about 1-6 hours, about 1-3 hours, or about 1-2 hours;

ii) median half-life (t_{1/2}) of Compound 1 about 1-3 hours or about 1-2 hours; and

iii) median area under the curve extrapolated to infinity (AUC_{inf}) of Compound 1 about 18 h*ng/ml to about 380 h*ng/mL.

In some embodiments, the subject experiences a reduction in at least one of body weight, blood pressure, and blood glucose after the administration.

In some embodiments, the subject experiences at least one of:
i) a reduction of body weight by at least 5% or 10%;
ii) a reduction of blood pressure of at least 5 mmHg;
iii) a reduction of HbA_{1c} by at least 0.5%, or at least 1.5%;
iv) a reduction of lipids by at least 10%;
v) a reduction of liver fat by at least 50%;
vi) a reduction of serum alanine aminotransferase (ALT); and
vii) a reduction of aspartate aminotransferase (AST).

### Compound 1

Compound 1 refers to 5-[(2,4-dinitrophenoxy)methyl]-1-methyl-2-nitro-1H-imidazole.

In some embodiments, Compound 1 is administered at about 30 mg, 100 mg, 200 mg, 500 mg, or 1050 mg per day.

In certain embodiments, the therapeutically effective amount is from about from about 30 mg to about 1400 mg per day, from about 50 mg to about 100 mg per day, from about 150 mg to about 600 mg per day, or from 200 mg to 550 mg per day.

In certain embodiments, the therapeutically effective amount is about 100 mg, 150 mg, 200 mg, 250 mg, 300 mg, 350 mg, 400 mg, 450 mg, 500 mg, or 600 mg per day.

In certain embodiments, the therapeutically effective amount is about 30 mg, 35 mg, 40 mg, 45 mg, 50 mg, 55 mg, 60 mg, 65 mg, 70 mg, 75 mg, 80 mg, 85 mg, 90 mg, or 95 mg, per day.

In certain embodiments, therapeutically effective amount is about 150 mg, 300 mg, or 450 mg per day.

In some embodiments, the therapeutically effective amount is chosen to adjust Cₘₐₓ, Tₘₐₓ and AUC.

### Pharmaceutical Salts

The compound of formula I may be used in its native form or as a salt. In cases where forming a stable nontoxic acid or base salt is desired, administration of the compound as a pharmaceutically acceptable salt may be appropriate.

Suitable pharmaceutically acceptable salts include prepared from inorganic and organic acids including sulfate, hydrogen sulfate, hydrochloric, hydrobromic, hydnodic, nitric, carbonic, sulfuric, phosphoric acids, formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, sulfanilic, stearic, alginic, 2-hydroxyethanesulfonic, p-toluene sulfonic, cyclohexylaminosulfonic, salicylic, galactaric, β-hydroxybutyric and galacturonic acid; or prepared from ammonium salts and metallic salts including calcium, magnesium, potassium, sodium and zinc salts.

### Composition/Formulation

Pharmaceutical compositions of the present disclosure may be manufactured by processes well known in the art, e.g., by means of conventional mixing, dissolving, granulation, dragee-making, levitating, emulsifying, encapsulating, entrapping, lyophilizing processes or spray drying.

Pharmaceutical compositions for use in accordance with the present disclosure may be formulated in conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant disclosure. Such excipients and carriers are described, for example, in “Remington's Pharmaceutical Sciences” Mack Pub. Co., New Jersey (1991).

In some embodiments, the pharmaceutical composition comprises Compound 1 or a pharmaceutically acceptable salt thereof, and a pharmaceutically acceptable excipient.

In some embodiments, the methods of the present disclosure comprise administering to the subject a pharmaceutical composition comprising compound 1 or a pharmaceutically acceptable salt thereof, and a pharmaceutically acceptable excipient.

### Pharmaceutical Dosage Forms

The present disclosure includes novel pharmaceutical dosage forms of Compound 1 or a pharmaceutically acceptable salt thereof. The dosage forms described herein are suitable for oral administration to a subject. The dosage form may be in any form suitable for oral administration, including, but not limited to, a capsule or a tablet. In some embodiments, the present disclosure provides a single unit dosage capsule or tablet form containing from about 30 mg to about 1400 mg, from about 100 mg to about 1000 mg, from about 150 mg to about 600 mg, or from 200 mg to 550 mg of Compound 1 or a pharmaceutically acceptable salt thereof. In some embodiments, Compound 1 is administered in a hydroxypropyl methylcellulose capsule.

In some embodiments, the amount of Compound 1 in a unit dosage is about 30 mg, 50 mg, 75 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg. In some embodiments, the single unit dosage form is a capsule. In some embodiments, the single unit dosage form is a tablet.

In some embodiments, the amount of Compound 1 in a unit dosage is about 30 mg, 40 mg, 50 mg, 60 mg, 70 mg, 75 mg, 80 mg, 90 mg, 100 mg, 150 mg, 170 mg, 200 mg, 250 mg, 300 mg, 340 mg, 350 mg, 400 mg, 450 mg, 500 mg, 510 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, or 1400 mg. In some embodiments, the single unit dosage form is a capsule. In some embodiments, the single unit dosage form is a tablet.

In some embodiments, the amount of Compound 1 in a unit dosage is about 30 mg, 100 mg, 200 mg, 500 mg, 600 mg, 1050 mg, or 1400 mg. In some embodiments, the amount of

Compound 1 in a unit dosage is about 200 mg, 400 mg, or 550 mg. In some embodiments, the amount of Compound 1 in a unit dosage is about 170 mg, 340 mg, 510 mg. In some embodiments, the amount of Compound 1 in a unit dosage is about 150 mg, 300 mg, 450 mg.

In some embodiments, the amount of Compound 1 in a unit dosage is about 30 mg, 35 mg, 40 mg, 45 mg, 50 mg, 55 mg, 60 mg, 65 mg, 70 mg, 75 mg, 80 mg, 85 mg, 90 mg, or 95 mg, per day. Routes of Administration

In therapeutic use for controlling or preventing weight gain in a mammal, a compound of the present disclosure or its pharmaceutical compositions can be administered orally, or parenterally.

In certain embodiments, the compound of the present disclosure or its pharmaceutical compositions can be administered once daily orally.

### Examples

| Abbreviation | Description |
|---|---|
| AUC₀₋ₜ | Area under the curve (AUC) from the time of |
| | dose administration to a specified time (t). |
| AUC_{inf} | Area under the curve (AUC) extrapolated to infinity. |
| Cₘₐₓ | Maximum concentration occurring at Tₘₐₓ. |
| Tₘₐₓ | First time to maximum observed concentration |
| | sampled during a dosing interval. |
| t_{1/2} | Terminal half-life. |
| ALT | Serum alanine aminotransferase |
| AST | Aspartate aminotransferase |
| BMI | Body mass index (kg/m²) |
| HbA1c | Glycosylated hemoglobin (hemoglobin A1c) |
| HBsAg | Hepatitis B surface antigen |
| HCV | Hepatitis C virus |
| HCVab | Hepatitis C virus antibody |
| HDL | High-density lipoprotein |
| HDPE | High-density polyethylene |
| HIV | Human immunodeficiency virus |
| HPMC | Hydroxypropyl methylcellulose |
| hs-CRP | High sensitivity C-reactive protein |
| MAD | Multiple ascending dose |
| MCHC | Mean corpuscular hemoglobin concentration |
| MCV | Mean cell volume |
| MRI | Magnetic resonance imaging |
| MRI-PDFF | Magnetic resonance imaging proton density fat fraction |
| NAFLD | Nonalcoholic fatty liver disease |
| NASH | Nonalcoholic steatohepatitis |
| OCT | Optical coherence tomography |
| OHS | Obesity hypoventilation syndrome |
| OSA | Obstructive sleep apnea |
| OWL | One Way Liver |
| PCP | Phencyclidine |
| PD | Pharmacodynamic(s) |
| PEth | Phosphatidylethanol |
| PIIINP | Procollagen III amino-terminal peptide |
| PK | Pharmacokinetic(s) |
| PPK | Population pharmacokinetic(s) |
| QD | Once daily |
| QTcF | QT interval corrected for heart rate using Fridericia cubed |
| RMR | Resting metabolic rate |
| SAD | Single ascending dose |
| SAE | Serious adverse event |
| SAP | Statistical analysis plan |
| SGLT2 | Sodium-glucose cotransporter-2 |
| T4 | Thyroxine |
| TdP | Torsades de pointes |
| TIMP-1 | Tissue inhibitor of matrix metalloproteinase 1 |
| Tmax | Time to peak plasma concentration |
| TSH | Thyroid stimulating hormone |
| TZD | Thiazolidinediones |
| UA | Urinalysis |
| ULN | Upper limit of normal |
| VCTE | Vibration-controlled transient elastography |
| VLDL | Very low-density lipoproteins |

### Example 1: Preclinical Studies

The therapeutic activity of Compound 1 was evaluated following oral administration in a mouse model of NASH (DIAMOND™ mice) and a rat model of metabolic syndrome (Zucker diabetic fatty rats). Compound 1 demonstrated efficacy in these models at a dose level of 5 mg/kg/day in mice and at ≥0.5 mg/kg/day in rats.

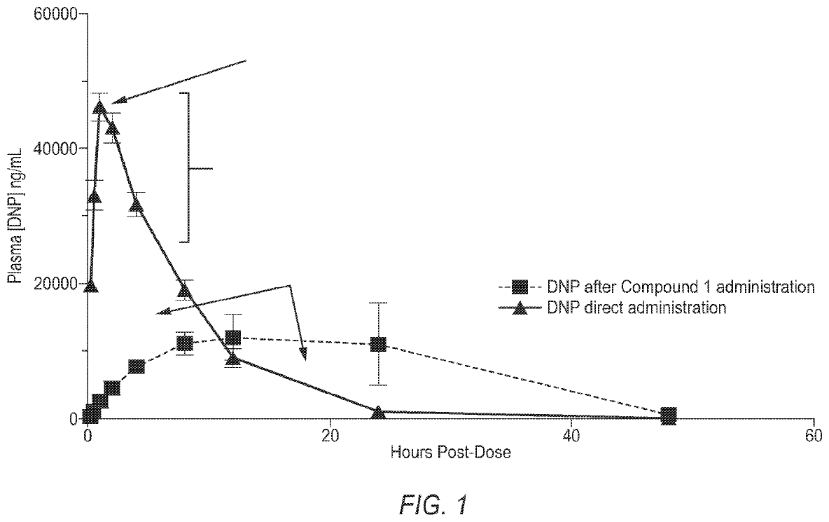

The PK of Compound 1 was evaluated in mice, rats, and dogs. Compound 1 was rapidly absorbed with conversion to 2,4-dinitrophenol in all species, and the results demonstrated that time to peak plasma concentration (Tₘₐₓ) for 2,4-dinitrophenol was substantially delayed following oral administration of Compound 1 when compared to direct oral administration of 2,4-dinitrophenol itself. FIG. 1 shows the plasma concentration of 2,4-dinitrophenol after administration of Compound 1 and 2,4-dinitrophenol.

The administration of Compound 1 also led to significantly higher AUC/Cₘₐₓ ratios for 2,4-dinitrophenol when compared to administration of 2,4-dinitrophenol directly. This optimized pharmacokinetic profile of 2,4-dinitrophenol following oral administration of Compound 1 led to substantial improvements in tolerability and safety evaluation in animals. In addition, the in vitro plasma protein binding of Compound 1 was tested in a number of species, and the tissue distribution and excretion following oral administration to rats were investigated. The metabolism of Compound 1 was evaluated in vitro using liver microsomes and hepatocytes from both nonclinical species and humans as well as using human recombinant metabolizing enzymes; these results led to the selection of the rat and dog as the rodent and non-rodent species for pivotal safety testing. Preliminary investigations into the drug-drug interaction potential of Compound 1 have not revealed the potential for drug-drug interactions.

Compound 1 was evaluated in a nonclinical toxicology program comprised of acute toxicity/tolerability, repeat-dose toxicity, and genotoxicity studies in accordance with International Council for Harmonisation (ICH) Guidance M3 (R2). All studies critical for supporting the safety evaluations were conducted in compliance with Food and Drug Administration (FDA) Good Laboratory Practices (GLP) regulations. Overall, the results of single-dose tolerability and repeat-dose toxicity studies in mice, rats, and dogs demonstrate that Compound 1 administration is well tolerated at doses up to 100/kg/day in mice (7-days), 120 mg/kg/day in rats (up to 63-days), and 100 mg/kg/day in dogs (up to 61 days).

There were no Compound 1-related deaths or effects on body weight, body weight gains, food consumption, body temperature, ophthalmology examination, electrocardiograms, hematology, clinical chemistry, or urinalysis (UA).

### Example 2: Single Ascending Dose (SAD) Study

A double-blind, placebo-controlled, phase I study of the safety and pharmacokinetics of single ascending doses of compound 1 in healthy volunteers was conducted. The volunteers were orally administered Compound 1 in doses ranging from 30 mg once daily (QD) to 1400 mg QD in a total of 67 subjects (50 active/17 placebo) across 8 cohorts. The objectives of this study are as follows:
To establish the pharmacokinetic profile of Compound 1 and 2,4-dinitrophenol in plasma in healthy volunteers, following single doses.
To establish the dose/exposure relationships for single doses with Compound 1, over a wide range of exposures in healthy volunteers.
To estimate the pharmacokinetic influence of a 50% fat meal concomitantly with Compound 1.
To characterize the pharmacokinetics of Compound 1 and 2,4-dinitrophenol.

Screening procedures occurred on Days -28 to -3. For all Cohorts except Cohort 4 (Fed/Fasted), subjects were admitted to the clinical research unit for up to 4 days. Administration of a single dose of study drug occurred on Day 1 in a fasted condition (8-hour fast), except for the crossover food effect cohort, which had a 10-hour fast prior to the meal. Following completion of all safety assessments and blood draws for PK analyses, subjects were discharged on Day 3. The Fed/Fasted cohort remained in the unit and were discharged on Day 7.

Double-blind dosing occurred in cohorts 1 through 8. In these cohorts, six subjects received Compound 1 and two received matching placebo. Doses escalated in the following sequence: 30, 100, 200, 500, 1400, 1050 and 600 mg.
Cohort 4 crossed-over to a fed state (50% fat meal) administration after pharmacokinetics and an inpatient washout had occurred for 72 hours after the first dose with a final dose.

Dose Escalation is shown below in Table 1.

TABLE 1

Number of Subjects

| Cohort | Unit dose of Compound 1 | Compound 1 | Placebo |
|---|---|---|---|
| 1 | 30 mg | 6 | 2 |
| 2 | 100 mg | 6 | 2 |
| 3 | 200 mg | 6 | 2 |
| 4 | 500 mg (Cross over to Fed) | 6 | 2 |
| 5 | 1050 mg | 6 | 2 |

Subjects were admitted to the clinical research unit on Day 1. Dosing took place on the morning of Day 1, following an 8-hour fast, except the fed cohort, which had a 10-hour fast prior to the meal. Blood draws for assessment of PK parameters occurred per schedule of assessments. IC were taken per schedule of assessments. Subjects were released on Day 3 following completion of all blood draws and safety assessments. The Fed/Fasted cohort remained in the unit and was discharged on Day 7.

### Schematic Study Design

SAD Cohort

| | Day −2 [symbol] to −3 | | | | | | | | |
|---|---|---|---|---|---|---|---|---|---|
| | Screening | Day −1 | Day 1 | Day 2 | Day 3 | Day 5 | | | [symbol] Day 12 |
| 2 sentinel | | Inpatient | | | | Follow up | | | Safety Meeting |
| subjects | | | Dose | | | | | | Prior to dose escalation |
| | | | PK through 48 hours | | | | | | |
| | | Day −28 to −3 | | | | | | | |
| | | Screening | | Day −1 | Day 1 | Day 2 | Day 3 | Day 5 | [symbol] Day 11 |
| | 6 remaining | | | Inpatient | | | | Follow up | Safety Meeting |
| | subjects | | | | Dose | | | | Prior to dose escalation |

PK through 48 hours

[symbol]

indicates data missing or illegible when filed

### Drug Administration

Compound 1 or matching placebo was administered orally as a single dose. All subjects except the fed cohort, were dosed in the morning after an 8-hour fast and remained in a semi-reclined position for 1 hour and fasting for 4 hours post-administration. Capsules were swallowed with 240 mL (8 fluid ounces) of room temperature water.

Cohort 4 was a crossover food effect analysis. Subjects enrolled in Cohort 4 were dosed 30 minutes following the beginning of a standardized (consisting of 50% fat) meal (following a 10-hour fast) to evaluate for food effects. The study drug was administered with room temperature 240 mL (8 fluid ounces) of water.

The study shows that the 2,4-dinitrophenol curve is extended for all the cohort. Compound 1 maintains the efficacy of 2,4-dinitrophenol while suppressing the toxicity by flattening the Cₘₐₓ curve, providing “trickle like” effect for near 24/7 delivery.

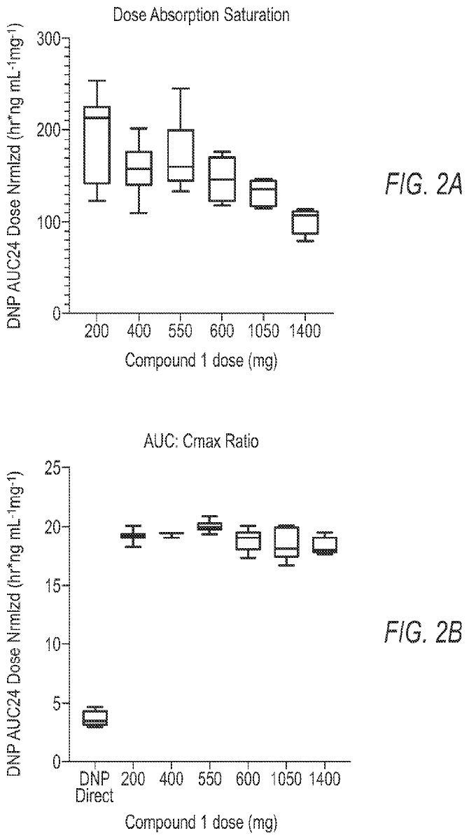

FIGS. 2A and 2B show the AUC of 2,4-dinitrophenol after Compound 1 administration. It demonstrates a saturable absorption of 2,4-dinitrophenol after administration of Compound 1. Thus, an overdose of 2,4-dinitrophenol can be prevented.

Pharmacokinetics of Compound 1 and 2,4-dinitrophenol

Compound 1 was dosed at 30 mg, 100 mg, 200 mg, 500 mg, and 1050 mg in the fasted state.

Compound 1 was absorbed rapidly with a median Tₘₐₓ from 1.75 to 3.00 hours and a median T_{lag}<0.50 hours across all dose levels. The mean t_{1/2} was short and ranged from 1.01 hours to 2.32 hours in the 500 mg and 1050 mg fasted cohorts. Mean apparent clearance and volume of distribution appear to increase with increasing dose. Exposures of Compound 1, based on dose-normalized Cₘₐₓ and AUC, appear to increase in a dose proportional manner between the 30 mg to 200 mg dose groups and less than dose proportional at doses greater than 200 mg (500 mg and 1050 mg).

2,4-Dinitrophenol appeared quickly after Compound 1 administration with a median T_{lag}<0.50 hours and a median Tₘₐₓ ranging from 6.01 hours to 10 hours. The mean t_{1/2} was relatively long and ranged from 30.0 hours to 38.4 hours across the dose levels. Mean apparent clearance and volume of distribution were higher for the 500 mg and 1050 mg dose groups relative to the 30, 100, or 200 mg dose groups. Exposures of 2,4-dinitrophenol, based on Cₘₐₓ and AUC, appear to increase in a less than dose proportional manner between the 30, 100, or 200 mg dose levels and the 500 or 1050 mg dose levels, with a 35-fold increase in dose showing a<18-fold increase in exposures.

### Example 3: Assessment of Food Effect

A single dose of 500 mg Compound 1 capsules in the fasted state was followed by a single dose of 500 mg Compound 1 capsules taken with a standardized (consisting of 50% high fat) meal (following a 10-hour fast). The geometric mean Compound 1 Cₘₐₓ was 14.8 ng/ml and 25.3 ng/ml with moderate variability of 52.1% and 49.3% geometric mean CV % respectively. The median Tₘₐₓ under fasted conditions was 3.0 hours and 6.0 hours under fed conditions. Geometric mean AUC₀₋₂₄ₕ was 53.5 h*ng/ml and 273 h*ng/ml with high to low variability of 86.5% and 7.92% geometric mean CV % respectively. Geometric mean AUCₗₐₛₜ was 44.3 h*ng/ml and 183 h*ng/ml with moderate variability of 66.2% and 44.3% geometric mean CV % for the fasted and fed groups respectively.

Compound 1 geometric mean ratio (90% CI) of Fed (test) versus Fasted (reference) for Cₘₐₓ was 1.82 (1.30-2.56), AUC₀₋₂₄ₕ was 5.11 (1.75-14.9), and for AUCₗₐₛₜ was 4.19 (2.75-6.39).

The geometric mean 2,4-dinitrophenol Cₘₐₓ was 694 ng/ml and 1680 ng/ml with moderate variability of 31.6% and 23.3% geometric mean CV % respectively. The median Tₘₐₓ under fasted conditions was 8.0 hours and 18.0 hours under fed conditions. Geometric mean AUC₀₋₂₄ₕ was 12400 h*ng/ml and 25300 h*ng/ml with moderate to low variability of 31.3% and 18.0% geometric mean CV % respectively. Geometric mean AUCₗₐₛₜ was 34200 h*ng/ml and 94000 h*ng/ml with moderate to low variability of 46.2% and 25.9% geometric mean CV % for the fasted and fed groups, respectively.

The 2,4-dinitrophenol geometric mean ratio (90% CI) of Fed (test) versus Fasted (reference) for Cₘₐₓ was 2.35 (1.85-2.99), AUC₀₋₂₄ₕ was 2.03 (1.53-2.70), and for AUCₗₐₛₜ was 2.58 (1.88-3.56).

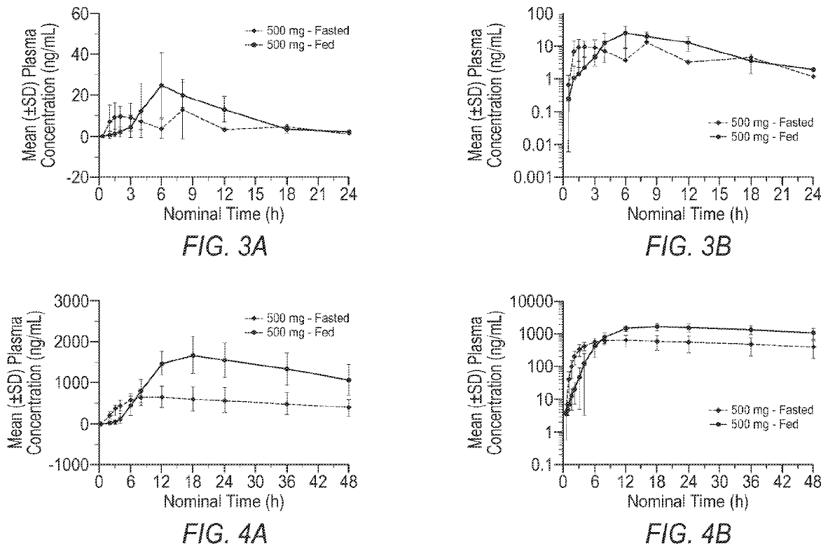

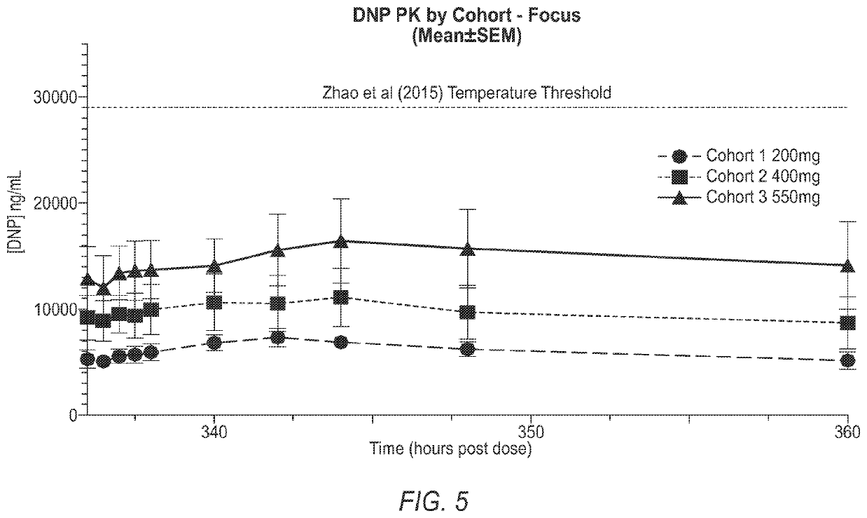

FIGS. 3a and 3b compares the plasma Compound 1 concentration by food status following 500 mg Compound 1 oral administration (Linear and Semi-log Scale). FIGS. 4a and 4b below compares the plasma 2,4-dinitrophenol concentration by food status following 500 mg Compound 1 oral administration (Linear and Semi-log Scale).

Summaries of the plasma pharmacokinetic parameters of Compound 1 after administration are shown in Table 2 below. Summaries of the plasma pharmacokinetic parameters of 2,4-dinitrophenol after administering Compound 1 are shown in Table 3 below.

TABLE 2

| | | T [symbol] | T [symbol] | Cₘₐₓ | C [symbol] /D | T [symbol] | AUC [symbol] | AUC [symbol] /D | AUC_{0-24 h} |
|---|---|---|---|---|---|---|---|---|---|
| Treatment | | (h) | (h) | (ng/mL) | (ng/mL/mg) | (h) | (h*ng/mL) | (h*ng/mL/mg) | (h*ng/mL) |
| 30 mg | N | 4 | 4 | 4 | 4 | 4 | 4 | 4 | 0 |
| Fasted | Mean (SD) | — | — | 1.86 (0.656) | 0.0618 (0.0219) | — | 4.53 (0.913) | 0.151 (0.0304) | — |
| | CV % | — | — | 35.4 | 35.4 | — | 20.1 | 20.1 | — |
| | Geometric Mean | — | — | 1.77 | 0.0589 | — | 4.46 | 0.149 | — |
| | Geometric CV % | — | — | 37.9 | 37.9 | — | 21.3 | 21.3 | — |
| | Median | 0.500 | 1.76 | 1.85 | 0.0615 | 4.00 | 4.59 | 0.153 | — |
| | Min, Max | 0.00, 0.517 | 1.00, 2.00 | 1.21, 2.52 | 0.0403, 0.0840 | 4.00, 6.00 | 3.36, 5.59 | 0.112, 0.186 | — |
| 100 mg | N | 6 | 6 | 6 | 6 | 6 | 6 | 6 | 1 |
| Fasted | Mean (SD) | — | — | 3.73 (1.52) | 0.0373 (0.0152) | — | 10.7 (4.27) | 0.107 (0.0427) | — |
| | CV % | — | — | 40.6 | 40.6 | — | 39.8 | 39.8 | — |
| | Geometric Mean | — | — | 3.46 | 0.0346 | — | 10.0 | 0.100 | — |
| | Geometric CV % | — | — | 46.0 | 46.0 | — | 41.9 | 41.9 | — |
| | Median | 0.500 | 1.75 | 3.96 | 0.0396 | 6.00 | 9.71 | 0.0971 | 18.8 |
| | Min, Max | 0.500, 2.00 | 1.50, 3.00 | 1.87, 6.01 | 0.0187, 0.0601 | 4.00, 6.00 | 5.39, 17.9 | 0.0639, 0.879 | 18.8, 18.8 |
| 200 mg | N | 6 | 6 | 6 | 6 | 6 | 6 | 6 | 2 |
| Fasted | Mean (SD) | — | — | 12.3 (5.29) | 0.0615 (0.0264) | — | 38.3 (12.8) | 0.191 (0.0642) | — |
| | CV % | — | — | 43.0 | 43.0 | — | 33.6 | 33.6 | — |
| | Geometric Mean | — | — | 11.3 | 0.0567 | — | 36.3 | 0.181 | — |
| | Geometric CV % | — | — | 47.0 | 47.0 | — | 38.5 | 38.5 | — |
| | Median | 0.500 | 1.75 | 11.4 | 0.0568 | 8.00 | 40.5 | 0.202 | 43.1 |
| | Min, Max | 0.250, 0.500 | 1.50, 4.00 | 6.37, 18.8 | 0.0319, 0.0940 | 6.00, 8.13 | 20.3, 53.4 | 0.101, 0.267 | 36.5, 49.8 |

| | | λ [symbol] | t_{1/2} | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/mg) | (%) | (L/h) | (L) |
| 30 mg | N | 0 | 0 | 0 | 0 | 0 | 0 | 0 |
| Fasted | Mean (SD) | — | — | — | — | — | — | — |
| | CV % | — | — | — | — | — | — | — |
| | Geometric Mean | — | — | — | — | — | — | — |
| | Geometric CV % | — | — | — | — | — | — | — |
| | Median | — | — | — | — | — | — | — |
| | Min, Max | — | — | — | — | — | — | — |
| 100 mg | N | 1 | 1 | 1 | 1 | 1 | 1 | 1 |
| Fasted | Mean (SD) | — | — | — | — | — | — | — |
| | CV % | — | — | — | — | — | — | — |
| | Geometric Mean | — | — | — | — | — | — | — |
| | Geometric CV % | — | — | — | — | — | — | — |
| | Median | 0.659 | 1.05 | 18.8 | 0.188 | 4.54 | 5330 | 8080 |
| | Min, Max | 0.659, 0.659 | 1.05, 1.05 | 18.8, 18.8 | 0.188, 0.188 | 4.54, 4.54 | 5330, 5330 | 8080, 8080 |
| 200 mg | N | 2 | 2 | 2 | 2 | 2 | 2 | 2 |
| Fasted | Mean (SD) | — | — | — | — | — | — | — |
| | CV % | — | — | — | — | — | — | — |
| | Geometric Mean | — | — | — | — | — | — | — |
| | Geometric CV % | — | — | — | — | — | — | — |
| | Median | 0.476 | 1.49 | 43.1 | 0.215 | 6.14 | 4760 | 9970 |
| | Min, Max | 0.408, 0.544 | 1.27, 1.70 | 36.5, 49.7 | 0.182, 0.248 | 5.57, 6.70 | 4030, 5480 | 9860, 10100 |

| | | T [symbol] | T [symbol] | C [symbol] | C [symbol] /D | T [symbol] | AUC [symbol] | AUC [symbol] /D | AUC [symbol] |
|---|---|---|---|---|---|---|---|---|---|
| Treatment | | (h) | (h) | (ng/mL) | (ng/mL/mg) | (h) | (h*ng/mL) | (h*ng/mL/mg) | (h*mg/mL) |
| 500 mg | N | 8 | 8 | 8 | 8 | 8 | 8 | 8 | 4 |
| Fasted | Mean (SD) | — | — | 16.5 (7.81) | 0.0329 (0.0156) | — | 53.8 (44.1) | 0.108 (0.0882) | 68.0 (60.4) |
| | CV % | — | — | 47.4 | 47.4 | — | 82.0 | 82.0 | 88.7 |
| | Geometric | — | — | 14.8 | 0.0297 | — | 44.3 | 0.0887 | 53.5 |
| | Mean | | | | | | | | |
| | Geometric | — | — | 52.1 | 52.1 | — | 66.2 | 66.2 | 86.5 |
| | CV % | | | | | | | | |
| | Median | 0.250 | 3.00 | 14.2 | 0.0284 | 6.00 | 37.8 | 0.0755 | 42.5 |
| | Min, Max | 0.250, 0.500 | 1.02, 8.00 | 8.61, 26.7 | 0.0172, 0.0534 | 6,00, 24.0 | 24.6, 158 | 0.0491, 0.316 | 29.1, 158 |
| 500 mg | N | 6 | 6 | 6 | 6 | 6 | 6 | 6 | 3 |
| Fed | Mean (SD) | — | — | 27.7 (13.6) | 0.0554 (0.0273) | — | 196 (78.0) | 0.393 (0.156) | 273 (22.1) |
| | CV % | — | — | 49.3 | 49.3 | — | 39.8 | 39.8 | 8.08 |
| | Geometric | — | — | 25.3 | 0.0505 | — | 183 | 0.365 | 273 |
| | Mean | | | | | | | | |
| | Geometric | — | — | 49.3 | 49.3 | — | 44.3 | 44.3 | 7.92 |
| | CV % | | | | | | | | |
| | Median | 1.00 | 6.00 | 25.1 | 0.0502 | 18.0 | 198 | 0.396 | 261 |
| | Min, Max | 0.250, 2.00 | 6.00, 12.0 | 12.7, 53.0 | 0.0254, 0.106 | 12.0, 24.0 | 108, 292 | 0.217, 0.584 | 260, 299 |
| 600 mg | N | 6 | 6 | 6 | 6 | 6 | 6 | 6 | 5 |
| Fasted | Mean (SD) | — | — | 90.0 (46.2) | 0.150 (0.0770) | — | 246 (117) | 0.410 (0.194) | 215 (91.9) |
| Micronized | CV % | — | — | 51.3 | 51.3 | — | 47.3 | 47.3 | 42.8 |
| | Geometric | — | — | 79.9 | 0.133 | — | 221 | 0.368 | 197 |
| | Mean | | | | | | | | |
| | Geometric | — | — | 59.1 | 59.1 | — | 58.0 | 58.0 | 51.6 |
| | CV % | | | | | | | | |
| | Median | 0.250 | 1.75 | 81.8 | 0.136 | 10.0 | 240 | 0.399 | 221 |
| | Min, Max | 0.00, 0.267 | 1.00, 6.00 | 37.6, 155 | 0.0627, 0.258 | 6.00, 12.1 | 92.9, 415 | 0.155, 0.691 | 95.1, 335 |

| | | λ [symbol] | t_{1/2} | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/mg) | (%) | (L/h) | (L) |
| 500 mg | N | 3 | 3 | 3 | 3 | 3 | 3 | 3 |
| Fasted | Mean (SD) | — | — | — | — | — | — | — |
| | CV % | — | — | — | — | — | — | — |
| | Geometric Mean | — | — | — | — | — | — | — |
| | Geometric CV % | — | — | — | — | — | — | — |
| | Median | 0.801 | 0.865 | 37.9 | 0.0757 | 2.19 | 13200 | 15200 |
| | Min, Max | 0.506, 0.868 | 0.799, 1.37 | 29.1, 47.2 | 0.0583, 0.0944 | 1.68, 8.89 | 10600, 17200 | 33200, 33900 |
| 500 mg | N | 3 | 3 | 3 | 3 | 3 | 3 | 3 |
| Fed | Mean (SD) | 0.204 (0.0304) | 3.45 (0.537) | 281 (17.6) | 0.561 (0.0353) | 5.82 (4.55) | 1790 (108) | 8920 (1800) |
| | CV % | 14.9 | 15.6 | 6.29 | 6.29 | 78.2 | 6.07 | 20.2 |
| | Geometric Mean | 0.203 | 3.42 | 280 | 0.560 | 4.83 | 1780 | 8800 |
| | Geometric CV % | 15.4 | 15.4 | 6.19 | 6.19 | 82.6 | 6.19 | 20.5 |
| | Median | 0.210 | 3.31 | 271 | 0.541 | 3.41 | 1850 | 8830 |
| | Min, Max | 0.172, 0.232 | 2.99, 4.04 | 270, 301 | 0.540, 0.602 | 2.98, 11.1 | 1660, 1850 | 7170, 10800 |
| 600 mg | N | 5 | 5 | 5 | 5 | 5 | 5 | 5 |
| Fasted | Mean (SD) | 0.693 (0.217) | 1.12 (0.487) | 215 (91.9) | 0.358 (0.153) | 1.31 (0.861) | 3360 (1790) | 5540 (3670) |
| Micronized | CV % | 31.3 | 43.5 | 42.8 | 42.8 | 65.6 | 53.2 | 66.3 |
| | Geometric Mean | 0.660 | 1.05 | 197 | 0.329 | 1.07 | 3040 | 4610 |
| | Geometric CV % | 38.8 | 38.8 | 51.6 | 51.6 | 85.4 | 51.6 | 76.7 |
| | Median | 0.749 | 0.926 | 221 | 0.368 | 0.888 | 2720 | 3680 |
| | Min, Max | 0.354, 0,908 | 0.764, 1.96 | 95.1, 335 | 0.159, 0.558 | 0.392, 2.33 | 1790, 6310 | 2160, 10500 |

| | | T [symbol] | T [symbol] | C [symbol] | C [symbol] /D | T [symbol] | AVC [symbol] | AUC [symbol] /D | AUC [symbol] |
|---|---|---|---|---|---|---|---|---|---|
| Treatment | | (h) | (h) | (ng/mL) | (ng/mL/mg) | (h) | (h*ng/mL) | (h*ng/mL/mg) | (h*ng/mL) |
| 1050 mg | N | 6 | 6 | 6 | 6 | 6 | 6 | 6 | 6 |
| Fasted | Mean (SD) | — | — | 17.1 (12.0) | 0.0163 (0.0115) | — | 69.1 (41.4) | 0.0658 (0.0394) | 71.7 (41.5) |
| | CV % | — | — | 70.2 | 70.2 | — | 59.9 | 59.9 | 57.9 |
| | Geometric | — | — | 14.6 | 0.0139 | — | 60.5 | 0.0576 | 63.2 |
| | Mean | | | | | | | | |
| | Geometric | — | — | 64.7 | 64.7 | — | 59.5 | 59.5 | 57.7 |
| | CV % | | | | | | | | |
| | Median | 0.250 | 2.50 | 12.7 | 0.0121 | 10.0 | 56.2 | 0.0535 | 58.1 |
| | Min, Max | 0.250, 0.500 | 1.00, 4.00 | 7.58, 40.2 | 0.00722, 0.0383 | 6.02, 18.0 | 31.8, 144 | 0.0303, 0.137 | 34.0, 145 |
| 1050 mg | N | 6 | 6 | 6 | 6 | 6 | 6 | 6 | 6 |
| Fasted | Mean (SD) | — | — | 131 (26.7) | 0.125 (0.0254) | — | 529 (445) | 0.504 (0.423) | 531 (443) |
| Micronized | CV % | — | — | 20.3 | 20.3 | — | 84.1 | 84.1 | 83.5 |
| | Geometric | — | — | 129 | 0.123 | — | 434 | 0.413 | 437 |
| | Mean | | | | | | | | |
| | Geometric | — | — | 21.4 | 21.4 | — | 68.3 | 68.3 | 67.8 |
| | CV % | | | | | | | | |
| | Median | 0.250 | 1.75 | 128 | 0.122 | 12.0 | 347 | 0.330 | 350 |
| | Min, Max | 0.00, 0.250 | 1.00, 6.02 | 90.3, 171 | 0.0860, 0.163 | 8.00, 24.0 | 266, 1420 | 0.254, 1.35 | 267, 1420 |
| 1400 mg | N | 6 | 6 | 6 | 6 | 6 | 6 | 6 | 6 |
| Fasted | Mean (SD) | — | — | 140 (31.5) | 0.0998 (0.0225) | — | 433 (110) | 0.310 (0.0782) | 429 (108) |
| Micronized | CV % | — | — | 22.6 | 22.6 | — | 25.3 | 25.3 | 25.1 |
| | Geometric | — | — | 136 | 0.0973 | — | 422 | 0.301 | 418 |
| | Mean | | | | | | | | |
| | Geometric | — | — | 26.6 | 26.6 | — | 25.8 | 25.8 | 25.1 |
| | CV % | | | | | | | | |
| | Median | 0.250 | 1.50 | 147 | 0.105 | 15.0 | 421 | 0.300 | 404 |
| | Min, Max | 0.250, 0.250 | 1.00, 3.00 | 83.4, 166 | 0.0596, 0.119 | 8.00, 36.0 | 320, 572 | 0.229, 0.409 | 322, 573 |

| | | λ [symbol] | t [symbol] | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/ng) | (%) | (L/h) | (L) |
| 1050 mg | N | 6 | 6 | 6 | 6 | 6 | 6 | 6 |
| Fasted | Mean (SD) | 0.390 (0.143) | 2.32 (1.81) | 72.0 (41.2) | 0.0686 (0.0392) | 4.92 (3.11) | 18400 (8710) | 62900 (59500) |
| | CV % | 36.8 | 78.3 | 57.2 | 57.2 | 63.2 | 47.4 | 94.6 |
| | Geometric Mean | 0.353 | 1.96 | 63.7 | 0.0606 | 3.87 | 16500 | 46700 |
| | Geometric CV % | 60.6 | 60.6 | 57.2 | 57.2 | 102 | 57.2 | 97.5 |
| | Median | 0.429 | 1.62 | 59.3 | 0.0565 | 5.28 | 18100 | 44900 |
| | Min, Max | 0.116, 0.502 | 1.38, 6.00 | 33.9, 145 | 0.0323, 0.138 | 0.963, 9.40 | 7230, 30900 | 15700, 180000 |
| 1050 mg | N | 6 | 6 | 6 | 6 | 6 | 6 | 6 |
| Fasted | Mean (SD) | 0.519 (0.263) | 1.71 (0.960) | 534 (449) | 0.509 (0.428) | 1.06 (0.842) | 2710 (1150) | 5640 (2610) |
| Micronized | CV % | 50.7 | 56.2 | 84.0 | 84.0 | 79.5 | 42.4 | 46.2 |
| | Geometric Mean | 0.461 | 1.50 | 438 | 0.417 | 0.736 | 2400 | 5200 |
| | Geometric CV % | 59.6 | 59.6 | 68.3 | 68.3 | 136 | 68.3 | 45.7 |
| | Median | 0.535 | 1.31 | 352 | 0.336 | 0.894 | 3010 | 4990 |
| | Min, Max | 0.218, 0.936 | 0.741, 3.18 | 267, 1440 | 0.254, 1.37 | 0.170, 2.27 | 731, 3930 | 3350, 10100 |
| 1400 mg | N | 5 | 5 | 5 | 5 | 5 | 5 | 5 |
| Fasted | Mean (SD) | 0.548 (0.300) | 1.73 (1.16) | 428 (121) | 0.306 (0.0863) | 0.547 (0.162) | 3480 (909) | 7720 (3630) |
| Micronized | CV % | 54.9 | 67.4 | 28.2 | 28.2 | 29.6 | 26.2 | 47.0 |
| | Geometric Mean | 0.474 | 1.46 | 415 | 0.296 | 0.525 | 3370 | 7120 |
| | Geometric CV % | 70.1 | 70.1 | 28.2 | 28.2 | 34.1 | 28.2 | 46.1 |
| | Median | 0.489 | 1.42 | 372 | 0.265 | 0.579 | 3770 | 6830 |
| | Min, Max | 0.191, 0.924 | 0.750, 3.63 | 322, 574 | 0.230, 0.410 | 0.308, 0.755 | 2440, 4350 | 4710, 13500 |

CV = Coefficient of variance, n = number of subjects with non-missing values, SD = Standard deviation, % CV = 100 × (SD/Mean), where SD and Mean are the standard deviation and arithmetic mean of untransformed data, respectively, Geometric % CV = 100 × Sq [symbol] (e [symbol] − 1), where SD is the standard deviation of natural log-transformed data. [symbol]
indicates data missing or illegible when filed

TABLE 3

| | | T [symbol] | T [symbol] | C [symbol] | C [symbol] /D | T [symbol] | AUC [symbol] | AUC [symbol] /D | AUC_{0/24 h} |
|---|---|---|---|---|---|---|---|---|---|
| Treatment | | (h) | (h) | (ng/mL) | (ng/mL/mg) | (h) | (h*ng/mL) | (h*ng/mL/mg) | (h*ng/ML) |
| 30 mg | N | 6 | 6 | 6 | 6 | 6 | 6 | 6 | 6 |
| Fasted | Mean (SD) | — | — | 81.0 (24.1) | 2.70 (0.803) | — | 3980 (1570) | 133 (52.2) | 1500 (448) |
| | CV % | — | — | 29.7 | 29.7 | — | 39.4 | 39.4 | 30.0 |
| | Geometric Mean | — | — | 77.8 | 2.59 | — | 3730 | 124 | 1440 |
| | Geometric CV % | — | — | 32.6 | 32.6 | — | 40.5 | 40.5 | 32.7 |
| | Median | 0.500 | 6.01 | 82.9 | 2.76 | 96.8 | 3430 | 114 | 1500 |
| | Min, Max | 0.500, 1.00 | 4.00, 18.0 | 47.6, 115 | 1.59, 3.83 | 48.1, 145 | 2620, 5850 | 87.4, 195 | 874, 2120 |
| 100 mg | N | 6 | 6 | 6 | 6 | 6 | 6 | 6 | 6 |
| Fasted | Mean (SD) | — | — | 248 (35.1) | 2.48 (0.351) | — | 11300 (2460) | 113 (24.6) | 4710 (711) |
| | CV % | — | — | 14.1 | 14.1 | — | 21.7 | 21.7 | 15.1 |
| | Geometric Mean | — | — | 246 | 2.46 | — | 11100 | 111 | 4660 |
| | Geometric CV % | — | — | 15.5 | 15.5 | — | 22.6 | 22.6 | 16.6 |
| | Median | 0.500 | 7.00 | 259 | 2.59 | 96.3 | 11500 | 115 | 4820 |
| | Min, Max | 0.250, 0.500 | 6.00, 12.0 | 184, 286 | 1.84, 2.86 | 48.0, 98.0 | 8420, 14200 | 84.2, 142 | 3410, 5560 |
| 200 mg | N | 6 | 6 | 6 | 6 | 6 | 6 | 6 | 6 |
| Fasted | Mean (SD) | — | — | 614 (102) | 3.07 (0.509) | — | 30000 (9110) | 150 (45.6) | 11200 (1970) |
| | CV % | — | — | 16.6 | 16.6 | — | 30.3 | 30.3 | 17.5 |
| | Geometric Mean | — | — | 607 | 3.04 | — | 29000 | 145 | 11100 |
| | Geometric CV % | — | — | 16.6 | 16.6 | — | 28.5 | 28.5 | 17.4 |
| | Median | 0.500 | 8.07 | 613 | 3.07 | 96.4 | 25500 | 127 | 10900 |
| | Min, Max | 0.250, 0.500 | 8.00, 12.0 | 493, 762 | 2.47, 3.81 | 95.2, 196 | 23100, 45000 | 115, 225 | 8850, 14300 |

| | | λ [symbol] | t_{1/2} | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/mg) | (%) | (L/h) | (L) |
| 30 mg | N | 3 | 3 | 3 | 3 | 3 | 3 | 3 |
| Fasted | Mean (SD) | 0.0188 (0.00485) | 38.4 (9.05) | 4420 (2460) | 147 (82.0) | 14.2 (4.52) | 8.04 (3.39) | 439 (211) |
| | CV % | 25.8 | 23.6 | 55.6 | 55.6 | 31.8 | 42.2 | 48.1 |
| | Geometric Mean | 0.0184 | 37.6 | 4030 | 134 | 13.8 | 7.44 | 404 |
| | Geometric CV % | 25.3 | 25.3 | 54.6 | 54.6 | 30.4 | 54.6 | 54.3 |
| | Median | 0.0173 | 40.1 | 3050 | 102 | 11.9 | 9.84 | 418 |
| | Min, Max | 0.0149, 0.0242 | 28.6, 46.4 | 2960, 7270 | 98.6, 242 | 11.4, 19.4 | 4.13, 10.1 | 239, 659 |
| 100 mg | N | 4 | 4 | 4 | 4 | 4 | 4 | 4 |
| Fasted | Mean (SD) | 0.0217 (0.00431) | 32.8 (5.69) | 13400 (3040) | 134 (30.4) | 13.8 (4.71) | 7.75 (1.70) | 364 (99.4) |
| | CV % | 19.8 | 17.4 | 22.7 | 22.7 | 34.1 | 21.9 | 27.3 |
| | Geometric Mean | 0.0214 | 32.3 | 13100 | 131 | 13.0 | 7.61 | 355 |
| | Geometric CV % | 18.8 | 18.8 | 22.8 | 22.8 | 43.4 | 22.8 | 25.0 |
| | Median | 0.0203 | 34.3 | 12900 | 129 | 15.4 | 7.87 | 320 |
| | Min, Max | 0.0184, 0.0279 | 24.8, 37.7 | 10600, 17100 | 106, 171 | 7.11, 17.3 | 5.85, 9.43 | 303, 512 |
| 200 mg | n | 5 | 5 | 5 | 5 | 5 | 5 | 5 |
| Fasted | Mean (SD) | 0.0243 (0.00597) | 30.0 (7.59) | 34100 (11700) | 171 (58.4) | 8.44 (4.94) | 6.35 (1.79) | 261 (39.1) |
| | CV % | 24.6 | 25.2 | 34.2 | 34.2 | 58.5 | 28.2 | 15.0 |
| | Geometric Mean | 0.0237 | 29.3 | 32700 | 164 | 6.98 | 6.12 | 258 |
| | Geometric CV % | 25.6 | 25.6 | 32.3 | 32.3 | 88.8 | 32.3 | 14.6 |
| | Median | 0.0232 | 29.9 | 27800 | 139 | 6.88 | 7.19 | 257 |
| | Min, Max | 0.0171, 0.0308 | 22.5, 40.5 | 24800, 52700 | 124, 264 | 2.07, 14.6 | 3.79, 8.08 | 221, 322 |

| | | T [symbol] | T [symbol] | C [symbol] | C [symbol] /D | T [symbol] | AUC [symbol] | AUC [symbol] /D | AUC_{0-24 h} |
|---|---|---|---|---|---|---|---|---|---|
| Treatment | | (h) | (h) | (ng/mL) | (ng/mL/mg) | (h) | (h*ng/mL) | (h*ng/mL/mg) | (h*ng/mL) |
| 500 mg | N | 8 | 8 | 8 | 8 | 8 | 8 | 8 | 8 |
| Fasted | Mean (SD) | — | — | 724 (234) | 1.45 (0.469) | — | 37200 (16700) | 74.4 (33.5) | 12900 (4040) |
| | CV % | — | — | 32.4 | 32.4 | — | 45.0 | 45.0 | 31.2 |
| | Geometric | — | — | 694 | 1.39 | — | 34200 | 68.4 | 12400 |
| | Mean | | | | | | | | |
| | Geometric | — | — | 31.6 | 31.6 | — | 46.2 | 46.2 | 31.3 |
| | CV % | | | | | | | | |
| | Median | 0.250 | 8.00 | 711 | 1.42 | 97.7 | 35200 | 70.4 | 12700 |
| | Min, Max | 0.250, 0.500 | 6.00, 18.0 | 426, 1220 | 0.852, 2.44 | 74.3, 122 | 16500, 72300 | 32.9, 145 | 7590, 21100 |
| 500 mg | N | 6 | 6 | 6 | 6 | 6 | 6 | 6 | 6 |
| Fed | Mean (SD) | — | — | 1720 (438) | 3.43 (0.875) | — | 96800 (27900) | 194 (55.7) | 25700 (5110) |
| | CV % | — | — | 25.5 | 25.5 | — | 28.8 | 28.8 | 19.9 |
| | Geometric | — | — | 1680 | 3.35 | — | 94000 | 188 | 25300 |
| | Mean | | | | | | | | |
| | Geometric | — | — | 23.3 | 23.3 | — | 25.9 | 25.9 | 18.0 |
| | CV % | | | | | | | | |
| | Median | 0.250 | 18.0 | 1580 | 3.16 | 98.7 | 84400 | 169 | 24500 |
| | Min, Max | 0.00, 1.50 | 12.0, 18.0 | 1370, 2540 | 2.74, 5.08 | 95.8, 149 | 74800, 150000 | 150, 299 | 21800, 35700 |
| 600 mg | N | 6 | 6 | 6 | 6 | 6 | 6 | 6 | 6 |
| Fasted | Mean (SD) | — | — | 4330 (2020) | 7.21 (3.37) | — | 213000 (98600) | 355 (164) | 79900 (34600) |
| Micronized | CV % | — | — | 46.7 | 46.7 | — | 46.3 | 46.3 | 43.3 |
| | Geometric | — | — | 3930 | 6.55 | — | 194000 | 323 | 73400 |
| | Mean | | | | | | | | |
| | Geometric | — | — | 52.4 | 52.4 | — | 52.6 | 52.6 | 48.7 |
| | CV % | | | | | | | | |
| | Median | 0.250 | 8.00 | 3880 | 6.47 | 124 | 214000 | 356 | 75000 |
| | Min, Max | 0.00, 0.267 | 4.00, 12.0 | 1890, 7280 | 3.15, 12.1 | 96.8, 148 | 90500, 376000 | 151, 626 | 36000, 132000 |

| | | λ [symbol] | t_{1/2} | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/mg) | (%) | (L/h) | (L) |
| 500 mg | N | 4 | 4 | 4 | 4 | 4 | 4 | 4 |
| Fasted | Mean (SD) | 0.0222 (0.00351) | 31.9 (5.42) | 32400 (13400) | 64.8 (26.9) | 11.5 (2.13) | 17.7 (7.65) | 780 (240) |
| | CV % | 15.8 | 17.0 | 41.5 | 41.5 | 18.5 | 43.2 | 30.8 |
| | Geometric Mean | 0.0219 | 31.6 | 30300 | 60.5 | 11.3 | 16.5 | 753 |
| | Geometric CV % | 16.6 | 16.6 | 45.2 | 45.2 | 20.3 | 45.2 | 31.3 |
| | Median | 0.0226 | 30.8 | 31200 | 62.4 | 12.2 | 16.4 | 735 |
| | Min, Max | 0.0176, 0.0259 | 26.7, 39.4 | 18000, 49200 | 36.0, 98.4 | 8.49, 13.1 | 10.2, 27.8 | 578, 1070 |
| 500 mg | N | 2 | 2 | 2 | 2 | 2 | 2 | 2 |
| Fed | Mean (SD) | — | — | — | — | — | — | — |
| | CV % | — | — | — | — | — | — | — |
| | Geometric Mean | — | — | — | — | — | — | — |
| | Geometric CV % | — | — | — | — | — | — | — |
| | Median | 0.0222 | 31.2 | 83800 | 168 | 6.56 | 5.97 | 269 |
| | Min, Max | 0.0219, 0.0225 | 30.8, 31.6 | 81800, 85800 | 164, 172 | 4.48, 8.65 | 5.83, 6.11 | 259, 279 |
| 600 mg | N | 5 | 5 | 5 | 5 | 5 | 5 | 5 |
| Fasted | Mean (SD) | 0.0232 (0.00656) | 31.8 (9.02) | 229000 (111000) | 382 (185) | 8.15 (5.82) | 3.20 (1.62) | 147 (84.1) |
| Micronized | CV % | 28.3 | 28.4 | 48.4 | 48.4 | 71.4 | 50.6 | 57.4 |
| | Geometric Mean | 0.0225 | 30.8 | 208000 | 346 | 5.54 | 2.80 | 128 |
| | Geometric CV % | 28.8 | 28.8 | 54.0 | 54.0 | 172 | 54.0 | 63.1 |
| | Median | 0.0236 | 29.4 | 219000 | 365 | 10.3 | 2.74 | 141 |
| | Min, Max | 0.0352, 0.0332 | 20.9, 45.5 | 107000, 390000 | 178, 650 | 0.861, 15.2 | 1.54, 5.62 | 64.5, 277 |

| | | T [symbol] | T [symbol] | C [symbol] | C [symbol] /D | T [symbol] | AUC [symbol] | AUC [symbol] /D | AUC_{0-24 h} |
|---|---|---|---|---|---|---|---|---|---|
| Treatment | | (h) | (h) | (ng/mL) | (ng/mL/mg) | (h) | (h*ng/mL) | (h*ng/nL/mg) | (h*ng/mL) |
| 1050 mg | N | 6 | 6 | 6 | 6 | 6 | 6 | 6 | 6 |
| Fasted | Mean (SD) | — | — | 1140 (631) | 1.09 (0.600) | — | 63500 (37000) | 60.5 (35.2) | 21500 (11600) |
| | CV % | — | — | 55.1 | 55.1 | — | 58.3 | 58.3 | 53.8 |
| | Geometric | — | — | 1030 | 0.979 | — | 56600 | 53.9 | 19300 |
| | Mean | | | | | | | | |
| | Geometric | — | — | 51.9 | 51.9 | — | 53.3 | 53.3 | 51.9 |
| | CV % | | | | | | | | |
| | Median | 0.250 | 10.0 | 854 | 0.813 | 100 | 47700 | 45.4 | 16300 |
| | Min, Max | 0.250, 0.500 | 6.02, 18.0 | 600, 2280 | 0.571, 2.17 | 96.6, 103 | 35000, 132000 | 33.3, 126 | 11800, 41200 |
| 1050 mg | N | 6 | 6 | 6 | 6 | 6 | 6 | 6 | 6 |
| Fasted | Mean (SD) | — | — | 8960 (3970) | 8.53 (3.78) | — | 461000 (233000) | 439 (222) | 162000 (59800) |
| Mi- | CV % | — | — | 44.3 | 44.3 | — | 50.6 | 50.6 | 36.9 |
| cronized | Geometric | — | — | 8410 | 8.01 | — | 426000 | 406 | 155000 |
| | Mean | | | | | | | | |
| | Geometric | — | — | 37.9 | 37.9 | — | 41.3 | 41.3 | 31.7 |
| | CV % | | | | | | | | |
| | Median | 0.250 | 601 | 7820 | 7.45 | 122 | 378000 | 360 | 147000 |
| | Min, Max | 0.00, 0.250 | 6.00, 24.0 | 6010, 16800 | 5.72, 16.0 | 97.1, 150 | 310000, 930000 | 296, 886 | 120000, 280000 |
| 1400 mg | N | 6 | 6 | 6 | 6 | 6 | 6 | 6 | 6 |
| Fasted | Mean (SD) | — | — | 8000 (1510) | 5.73 (1.08) | — | 413000 (162000) | 295 (116) | 143000 (18800) |
| Mi- | CV % | — | — | 18.9 | 18.9 | — | 39.2 | 39.2 | 13.2 |
| cronized | Geometric | — | — | 7880 | 5.63 | — | 388000 | 277 | 142000 |
| | Mean | | | | | | | | |
| | Geometric | — | — | 19.2 | 19.2 | — | 40.3 | 40.3 | 14.2 |
| | CV % | | | | | | | | |
| | Median | 0.250 | 6.00 | 8270 | 5.90 | 98.2 | 396000 | 283 | 149000 |
| | Min, Max | 0.00, 0.250 | 4.00, 36.0 | 6090, 10300 | 4.35, 7.36 | 95.4, 144 | 213000, 700000 | 152, 500 | 110000, 159000 |

| | | λ [symbol] | t_{1/2} | AUC [symbol] | AUC [symbol] /D | AUC [symbol] | CL/F | V [symbol] /F |
|---|---|---|---|---|---|---|---|---|
| Treatment | | ( [symbol] /h) | (h) | (h*ng/mL) | (h*ng/mL/mg) | (%) | (L/h) | (L) |
| 1050 mg | N | 5 | 5 | 5 | 5 | 5 | 5 | 5 |
| Fasted | Mean (SD) | 0.0203 (0.00151) | 34.3 (2.50) | 78600 (47200) | 74.8 (44.9) | 15.3 (1.52) | 16.8 (7.60) | 850 (417) |
| | CV % | 7.42 | 7.28 | 60.1 | 60.1 | 9.94 | 45.2 | 49.0 |
| | Geometric Mean | 0.0203 | 34.2 | 69300 | 66.0 | 15.2 | 15.2 | 748 |
| | Geometric CV % | 7.37 | 7.37 | 58.3 | 58.3 | 10.3 | 58.3 | 66.2 |
| | Median | 0.0198 | 35.0 | 57300 | 54.6 | 15.6 | 18.3 | 963 |
| | Min, Max | 0.0189, 0.0221 | 31.3, 36.7 | 41500, 156000 | 39.5, 148 | 12.9, 17.1 | 6.74, 25.3 | 304, 1280 |
| 1050 my | N | 6 | 6 | 6 | 6 | 6 | 6 | 6 |
| Fasted | Mean (SD) | 0.0201 (0.00100) | 34.6 (1.67) | 521000 (284000) | 497 (271) | 10.7 (5.67) | 2.33 (0.715) | 117 (37.3) |
| Micronized | CV % | 5.00 | 4.83 | 54.5 | 54.5 | 53.2 | 30.7 | 31.9 |
| | Geometric Mean | 0.0200 | 34.6 | 478000 | 455 | 9.30 | 2.20 | 110 |
| | Geometric CV % | 4.92 | 4.92 | 43.6 | 43.6 | 64.0 | 43.6 | 45.5 |
| | Median | 0.0197 | 35.1 | 422000 | 402 | 10.7 | 2.50 | 122 |
| | Min, Max | 0.0191, 0.0218 | 31.9, 36.3 | 364000, 1100000 | 347, 1040 | 4.70, 16.8 | 0.958, 2.89 | 46.5, 149 |
| 1400 mg | N | 4 | 4 | 4 | 4 | 4 | 4 | 1 |
| Fasted | Mean (SD) | 0.0262 (0.00348) | 26.8 (3.56) | 365000 (100000) | 261 (71.7) | 7.10 (2.92) | 4.12 (1.43) | 155 (35.5) |
| Micronized | CV % | 13.3 | 13.3 | 27.5 | 27.5 | 41.2 | 34.6 | 22.9 |
| | Geometric Mean | 0.0260 | 26.6 | 353000 | 252 | 6.66 | 3.96 | 152 |
| | Geometric CV % | 13.4 | 13.4 | 32.1 | 32.1 | 43.2 | 32.1 | 21.1 |
| | Median | 0.0260 | 26.8 | 391000 | 280 | 6.66 | 3.60 | 138 |
| | Min, Max | 0.0228, 0.0299 | 23.2, 30.4 | 225000, 453000 | 161, 324 | 4.18, 10.9 | 3.09, 6.21 | 135, 208 |

CV = Coefficient of variance, n = number of subjects with non-missing values, SD = Standard deviation, % CV = 100 × (SD/Mean), where SD and Mean are the standard deviation and arithmetic mean of untransformed data, respectively, Geometric % CV = 100 × Sq [symbol] (e [symbol] − 1), where SD is the standard deviation of natural log-transformed data. [symbol]
indicates data missing or illegible when filed

In the completed SAD study, Compound 1 was rapidly absorbed and converted to a saturable level of 2,4-dinitrophenol with a mean half-life ranging from 1-3 hours. 2,4-Dinitrophenol levels were significantly higher with a mean half-life less than 40 hours. A positive food effect on Compound 1 absorption was evident as was the impact of Compound 1 particle size on absorption as two formulations of Compound 1 of different particle size were evaluated. At high single doses, there was evidence of saturation of absorption. The AUC/Cₘₐₓ ratio was approximately 18 regardless of dose.

Single oral doses of 30-1400 mg Compound 1 were observed to be safe and well tolerated in all subjects in the SAD study. There were no Serious Adverse Events (SAEs) reported. There has been no demonstrable correlation between dose and adverse event incidence. The most represented System Organ Class (SOC) for adverse events (AEs) was Gastrointestinal, primarily consisting of lower abdominal discomfort and loose stools/diarrhea, and a higher incidence was observed in Compound 1 treated subjects than in placebo subjects. All AEs were mild or moderate in intensity with a majority of them assigned to mild. Review of ECGs, Vital Signs and Laboratory evaluation did not demonstrate any trends to abnormal findings or any relationship to dose. Discolored urine, described as green in color, noted in some subjects at higher dose levels appears to be a benign phenomenon.

The SAD study demonstrates that
Compound 1 pharmacokinetics are characterized by rapid absorption and a fast elimination.
2,4-Dinitrophenol appears quickly and has a relatively slow elimination.
The metabolite is formed quickly, has a relatively slow elimination, and circulates at low levels compared to 2,4-dinitrophenol (<1%).
A standard high fat (50%) meal administration delays the absorption of Compound 1 and appearance of 2,4-dinitrophenol while increasing total exposure (Cₘₐₓ and AUC) of Compound 1 and 2,4-dinitrophenol. Compound 1 and 2,4-dinitrophenol Cₘₐₓ increased by >1.8-fold and Compound 1 and 2,4-dinitrophenol AUC increased by >4.0-fold and >2.0-fold, respectively, under fed conditions relative to fasted conditions.

### Example 4: Multiple Ascending Dose (MAD) Study

A double-blind, sponsor-open, placebo-controlled, phase I study of the safety, pharmacokinetics and pharmacodynamics of multiple ascending doses of Compound 1 in high MBI volunteers was conducted. The first cohort of 10 healthy high body mass index (BMI) subjects has completed dosing at 200 mg Compound 1. Two more cohorts are also planned at doses of 400 and 550 mg QD.

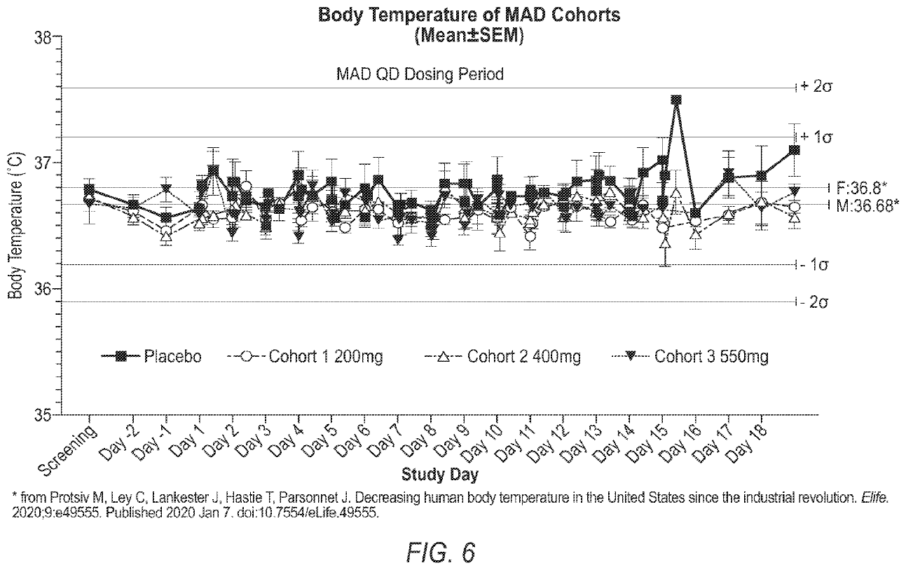

The subjects have tolerated oral Compound 1 at doses of 200 mg QD well. There have been no SAEs, and preliminary evaluation of the data has demonstrated no significant changes in physical examination, vital signs, ECGs, or laboratory data. FIG. 6 also shows that the 2,4-dinitrophenol curve is flat for all the cohorts, reaching a steady state of plasma concentration of 2,4-dinitrophenol.

FIG. 6 shows that there is no significant increase in body temperature after administering Compound 1.

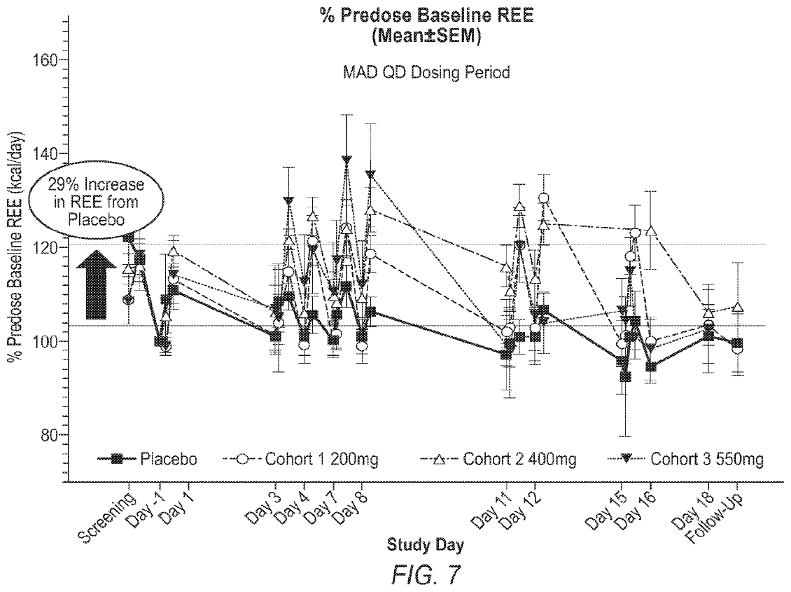

FIG. 7 below shows that Compound 1 increases resting energy expenditure (REE) about 29% compared to placebo.

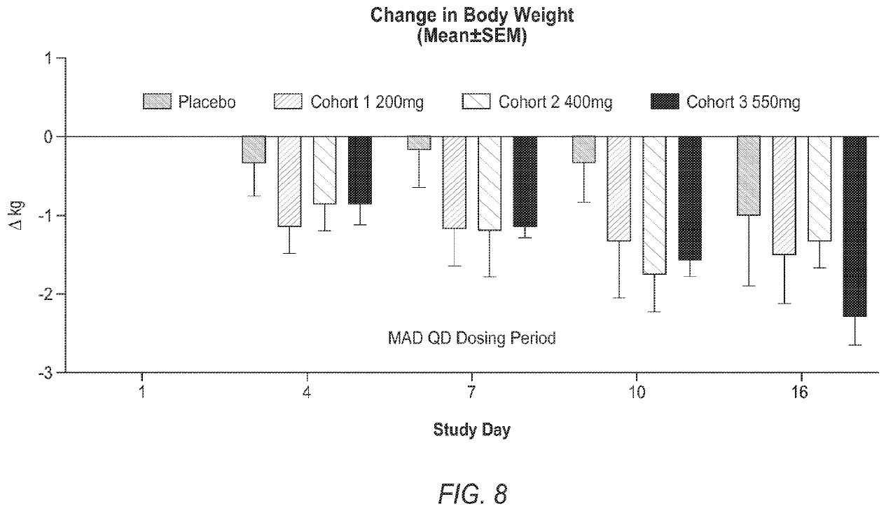

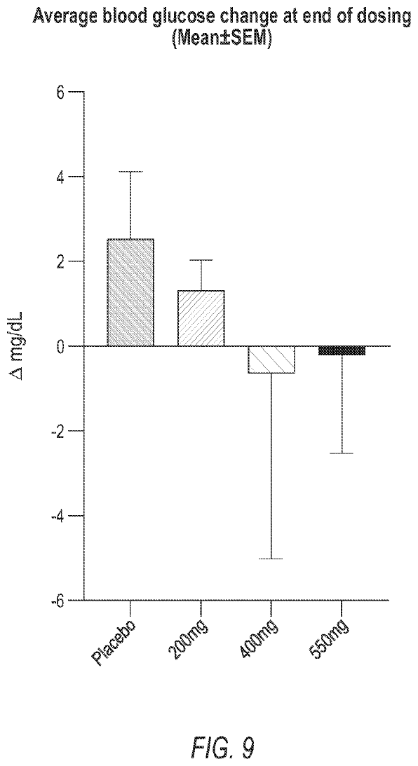

FIG. 8 and FIG. 9 show that Compound 1 reduces body weight and glucose level after administering Compound 1.

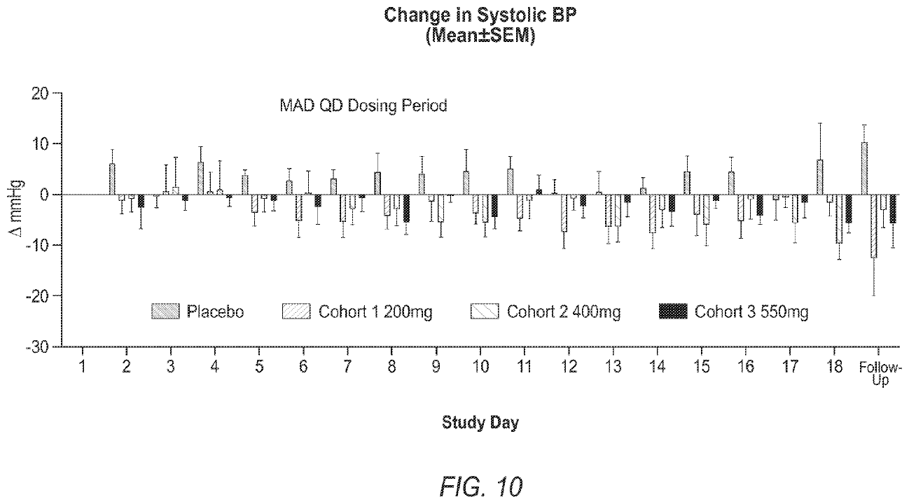

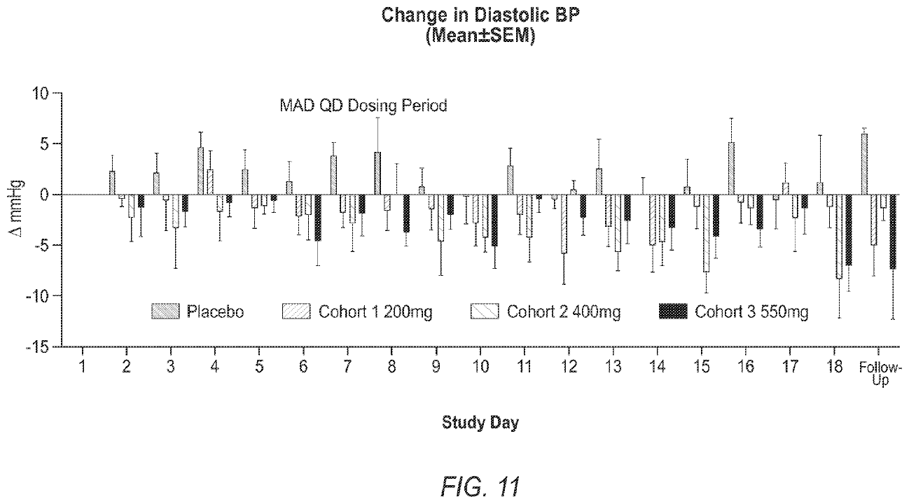

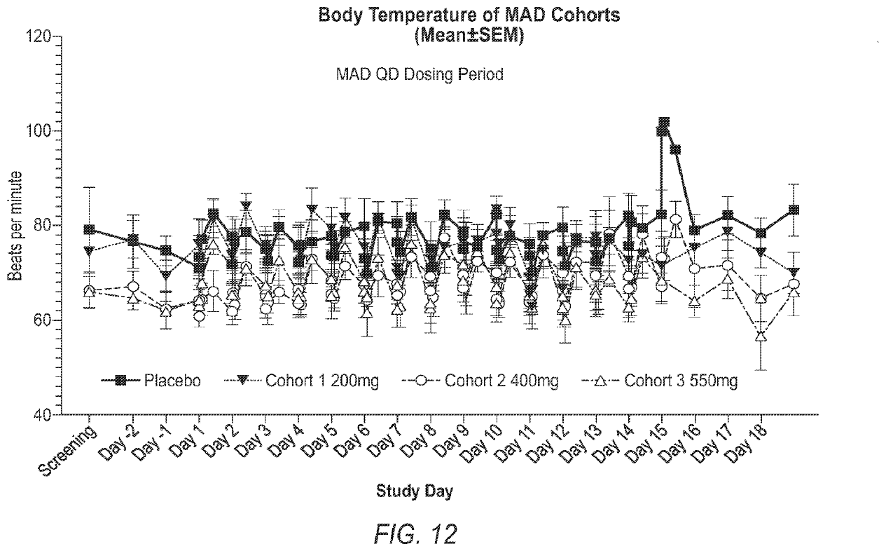

FIGS. 10 and 11 show that Compound 1 reduces systolic blood pressure and diastolic blood pressure after administering Compound 1, which leads to significant risk reduction in patients with cardiovascular diseases, such as HFpFF, HFrEF, and HFmrEF. The results show that the reductions in blood pressure are highly statistically significant, and changes were seen at all three dose levels. Almost immediate, rapid effect on both diastolic and systolic blood pressure was observed. The reduction in blood pressures, in conjunction with blood glucose and reduced adiposity, is strong indicative of Compound 1 being efficacious in HFpEF. FIG. 12 also indicates that the reductions in blood pressures and blood glucose as well as the reduced adiposity are not accompanied with an increase in heart rate.

### Example 5: Phase 2a Study of Compound 1 in Subjects with Elevated Liver Fat and High BMI

A 61-day randomized, double blind, placebo-controlled trial to assess the safety and efficacy of three doses of Compound 1 in subjects with elevated liver fat and high body mass index (28 to 45 kg/m²) is ongoing.

### Rationale for Study

High BMI subjects with evidence of elevated liver fat better replicate the metabolic characteristics of the eventual patient population with NASH (Golabi et al., 2020). Given the primacy of dysmetabolism in NASH pathogenesis, understanding safety and therapeutic effect of Compound 1 on liver fat primarily, and secondarily on body weight, in high BMI volunteers with elevated liver fat will provide greater confidence for potential efficacious dose selection for phase 2b clinical studies. Establishing doses and their associated pharmacokinetic exposures that lead to significant reductions in liver fat (a reduction≥50% in relative liver fat) in a 61-day study is anticipated to provide safety, target engagement and efficacy data for a longer-term (9 months) phase 2b in biopsy-verified NASH patients. Additionally, clinical effects of mitochondrial uncoupling were studied in populations reflecting similar metabolic characteristics of obesity (Tainter et al., 1934; Harper et al, 2001), providing further historical safety precedent for the patient population under study.

In aggregate, understanding safety and efficacy of Compound 1 on liver fat and body weight in a subject population that possesses the metabolic characteristics of a NASH patient population (high BMI and elevated liver fat) will provide greater confidence in dose selection and effect for subsequent longer-term clinical studies in phase 2b.

### Rationale for Selected Doses

All single dose exposures of Compound 1 and 2,4-dinitrophenol had an acceptable AE profile in the SAD study. Whereas Compound 1 has a short half-life, 2,4-dinitrophenol half-life is approximately 40 hours. With once daily dosing, significant accumulation of 2,4-dinitrophenol is anticipated at steady state. From cohort 1 of the MAD study, 200 mg QD for 14 days resulted in a dose accumulation factor of approximately 3.5-fold, reaching steady-state after 7 days of daily dosing.

Pharmacodynamic effects of single doses of Compound 1 in the SAD study were monitored using Indirect calorimetry (IC) to ascertain changes in resting metabolic rate (RMR). At single dose exposures of Compound 1 at doses as high as 1400 mg in the SAD study, a maximum increase of approximately 20% in RMR was found throughout the first 33 hours of IC assessments following dosing.

Given the safety of single dose exposures and the associated pharmacological effect on RMR, a starting repeat oral dose of 200 mg daily was anticipated to increase RMR by approximately 10%. This increase in RMR was confirmed in cohort 1 of the MAD study where an approximate 10% increase in RMR was evident by Day 7 of oral dosing of 200 mg daily and was sustained to day 15 of repeat dosing. Exposures at steady state of the 200 mg QD dose were as expected given dose accumulation. Given that the 200 mg QD dose had an acceptable safety profile after 2 weeks of dosing, the next cohort in the MAD study is being dosed with 400 mg QD of Compound 1 for 14 days. The anticipated increase in RMR is 20%, and dose exposures at steady state are projected to be within the range of the highest exposures achieved in the SAD study. Should the 400 mg QD dose have an acceptable safety profile after 2 weeks of dosing, the subsequent cohort is projected to be dosed with 550 mg QD of Compound 1, but the decision will be dependent upon the emerging data reviewed in real time. The anticipated increase in RMR at that projected dose/exposure is 30%, well within the impact of a meal on RMR and below what the historical clinical studies of 2,4-dinitrophenol found to have an acceptable safety profile. Given the approximately 40-hour half-life of 2,4-dinitrophenol, steady state exposures of 550 mg QD Compound I will be higher than single dose exposures measured in the SAD study. Population pharmacokinetic (PPK) modeling of Compound 1 and 2,4-dinitrophenol concentrations indicates that steady state 2,4-dinitrophenol concentrations achieved with daily dosing of 550 mg Compound 1 will still be below levels observed in clinical and nonclinical studies that impact body temperature. Historically 2,4-dinitrophenol has been associated with adverse increases in body temperatures in humans at concentrations greater than 28,000 ng/ml (Zhao 2015), and increases in body temperature, panting, and erythema were noted in studies with Compound 1 in dogs where plasma concentrations of 2,4-dinitrophenol exceeded 13,000 ng/mL. Additionally, an increase of approximately 200% in RMR anticipated an increase in body temperature (Bachynsky 2015 [US20150056160A1]). Thus, the expected Cₘₐₓ and steady state 2,4-dinitrophenol concentrations and resulting increase in RMR with Compound 1 dosing at 550 mg QD are well below the 2,4-dinitrophenol Cₘₐₓ and increase in RMR that are associated with increases in body temperature.

The study duration of 61 days was selected based on duration of dosing from completed toxicology studies with Compound 1 and to reflect the clinical experience with 2,4-dinitrophenol from studies conducted by Maurice Tainter as well as by Samuel Simkins (Tainter et al., 1934; Harper et al., 2001; Geisler, 2019). In these studies, 2,4-dinitrophenol was dosed from 1-3 months once daily at doses that readily caused a 20-40% increase in RMR. The resulting weight loss experienced ranged from 1.4-2.1 lbs./week. The drug was well tolerated at these dose levels over the course of 1-3 months. Day 61 of treatment, Compound 1 is anticipated to induce significant liver fat loss in concert with reduction in body weight and dose related RMR increases ranging from 10-40%. For example, 150 mg Compound 1 for 10% increase in resting energy expenditure; 300 mg Compound 1 for 20% increase in resting energy expenditure; and 450 mg Compound 1 for 30% increase in resting energy expenditure. The resulting safety and efficacy data Day 61 should provide significant guidance for dose selection for longer term Phase 2b studies.

### Primary Objectives

### Efficacy:

To evaluate the reduction in liver fat content, as assessed by magnetic resonance imaging proton density fat fraction (MRI-PDFF) from baseline to Day 61 in subjects with elevated BMI treated with Compound 1 compared to placebo.

### Safety:

To evaluate safety and tolerability of 61 days of repeated daily dosing of Compound 1 in overweight and obese subjects as defined by BMI.

### Secondary Objectives

To assess the rate and amount of body weight loss after 61 days of Compound 1 treatment.
To assess change from baseline in whole body adiposity by MRI after 61 days of Compound 1 treatment.
To characterize the PK profile of Compound 1 and 2,4-dinitrophenol over 61 days of dosing in subjects with high BMI.
To evaluate and correlate changes from baseline in measures of liver composition with changes in liver fat content after dosing with Compound 1.
To investigate the pharmacodynamic (PD) effects of Compound 1 on metabolic and cardiovascular risk factors.
To investigate the PD effects of Compound 1 on metabolomic, proteomic, and lipidomic profiles.
To characterize the dose/exposure relationships of the efficacy and PD effects of Compound 1, as data allow.

### Endpoints

### Primary Endpoint

Relative change from baseline in liver fat content, as assessed by MRI-PDFF at Day 61.

### Secondary Endpoints

### Pharmacodynamics

Change from baseline in body weight at Day 61.
Change from baseline in whole body adiposity at Day 61.
Changes from baseline in surrogate measures of liver inflammation and fibrosis at Day 61: Fibroscan Vibration-controlled Transient Elastography (VCTE), Fibroscan® Controlled Attenuation Parameter (CAP) score, and Enhanced Liver Fibrosis (ELF) score.
Change from baseline in lipid parameters and cardiovascular risk biomarkers at Day 61: serum high sensitivity C-reactive protein (hs-CRP), Lp (A), Apo B, low-density lipoprotein (LDL), high-density lipoprotein (HDL), total cholesterol, triglycerides, and free fatty acids (FFA).
Change from baseline in metabolic disease parameters at Day 61: homeostatic model assessment for insulin resistance (HOMA-IR), fasting blood glucose concentrations, glycated albumin concentrations, HbA_{1c}.

### Safety and Tolerability.

Summary of physical examinations findings over the course of the study.
Adverse event (AE) assessment over the course of the study.
Assessment of vital sign parameters over the course of the study. Parameters to include resting systolic and diastolic blood pressures, resting heart rates, resting respiratory rates, and oral body temperatures.
Assessment of body weight over the course of the study.
Safety 12-lead ECGs over the course of the study.
Assessment of clinical laboratory values (hematology, full biochemistry panel (including lipid panel, CPK, magnesium, liver function tests) and urinalysis (UA) over 61 days of dosing.
Assessment of ophthalmologic examination, including slit lamp, prior to and after 61 days of dosing.

### Pharmacokinetics

Population PK analysis of Compound 1 and 2,4-dinitrophenol. The following PK parameters will be estimated as appropriate: Cₘₐₓ, Tₘₐₓ, t_{1/2}, T_{lag}, AUC₀₋ₜ, AUC₀₋ₓ, CL/F, Vd/F, Az. Other PK parameters may be calculated, as data allow and appropriate.
As data permit, non-compartmental analysis of Compound 1 and 2,4-dinitrophenol. The following PK parameters will be calculated: Cₘₐₓ, AUC, and accumulation.
Modeling of exposure response relationships of Compound 1 and 2,4-dinitrophenol, and efficacy/pharmacodynamic endpoints, as appropriate.

### Exploratory Endpoints

Change from baseline in metabolomic and lipidomic profiles (One Way Liver-[OWL] metabolomic and lipidomic assays) at Day 61.
Change from baseline in proteomic profiles (SomaScanR) at Day 61.
Change from baseline in serum alanine aminotransferase (ALT) and aspartate aminotransferase (AST) concentrations at Day 61, as data allow.

### Study Design

This is a Phase 2a, randomized, parallel-group, placebo-controlled, double-blind, repeated-dose study to evaluate the safety and efficacy of three oral dose levels of Compound 1 compared to placebo over the course of 61 days in subjects with high BMI and evidence of elevated liver fat, as shown in the Scheme below:

Subjects will be screened over a 45-day period to determine their eligibility based on specific history, physical, laboratory and imaging evaluations. Due to scheduling of the procedures, multiple visits will likely be necessary to complete the screening process. However, if all screening assessments and procedures, including the MRI, can be completed within 30 days of the first dose, then a single screening visit is permissible.

Once qualified, patients will be randomly assigned to one of the Compound 1 treatment groups or the matched placebo control group and dosed once daily (fasting) for a total of 61 days. Subjects will return to the clinic for frequent assessment visits during the 61 days of dosing. A follow-up visit will occur within 10 to 14 days following the completion of dosing.

Subjects will be instructed to maintain their same diet and activity/exercise level throughout the study as they had prior to participation in the study.

Dosing will take place once daily in a fasted state. Eligible subjects will be randomized equally (N=20 per group) to 1 of 4 treatment groups:
Group 1: matched placebo orally once daily for 61 days.
Group 2:150 mg Compound 1 orally once daily for 61 days.
Group 3:300 mg Compound 1 orally once daily for 61 days.
Group 4:450 mg Compound 1 orally once daily for 61 days.

The randomization will be blocked and stratified by HbA_{1c} (normal range versus between 5.7% and 9.0% inclusive).

### Inclusion Criteria

Subjects must meet all the following inclusion criteria to be eligible:
1. Adult male or females, 28 to 65 years of age (inclusive) at the time of informed consent with BMI between 28.0 and 45.0 kg/m² (inclusive).
   a. Female subjects of childbearing potential must be non-lactating, not pregnant as confirmed by a negative urine pregnancy test at Screening and agree to continue using an effective method of contraception for at least 4 weeks or barrier method for 2 weeks prior to first study drug administration until 30 days after the last dose of study drug.
   b. Female subjects of childbearing potential must not donate ova during the study and for at least 30 days after the last dose of study drug.
   c. Female subjects of non-childbearing potential must be surgically sterile (e.g., hysterectomy, bilateral tubal ligation, oophorectomy) or postmenopausal (no menses for >1 year with follicle stimulating hormone (FSH)>40 U/L at Screening).
   d. Male subjects who have not had a vasectomy and/or subjects who have had a vasectomy but have not had 2 post surgery negative tests for sperm must agree to use an acceptable method of contraception from time of first dose of study drug until 30 days after the last dose of the study drug, and to not donate sperm during the study and for at least 30 days after the last dose of study drug.
2. Inclusion as per investigator assessment of general medical status and as documented by medical history, physical examination, vital sign assessments, 12-lead ECG, clinical laboratory assessments, and general observations.
   a. Subjects must be on stable doses of medications for underlying obesity-related conditions for at least 2 months prior to screening.
   b. Subjects with diabetes may be treated with metformin, DPP-4 inhibitors, or sulfonylureas, but must be on stable doses for at least 2 months prior to screening.
   c. At Screening, certain laboratory values may be outside the reference range if commensurate with the underlying obesity or associated metabolic dysfunction in the eligible subject (for example, dyslipidemia and hyperglycemia), unless these abnormalities suggest an underlying condition which may impact subject safety in the trial or interfere with the evaluation of Compound 1 or affect interpretation of the study results.
   d. Abnormalities or deviations outside the normal ranges for other assessments that are considered clinically significant by the Investigator (clinical laboratory tests, ECG, vital signs, physical examination) may be repeated once at the discretion of the Investigator(s). Results that continue to be outside the normal ranges must be judged by the investigator to be not clinically significant and acceptable for study participation.
   e. Subjects with elevation of unconjugated bilirubin due to presumptive Gilbert's syndrome are permissible.
   f. Subject must be euthyroid as assessed by a thyroid profile utilizing thyroid stimulating hormone (TSH) and free thyroxine (T4) testing at screening. Subjects with a stable history of thyroid disease and who have been on stable doses of thyroid medications for a minimum of 4 months can be enrolled.
3. Fibroscan® CAP score>300 dB/m.
≥8% liver fat by MRI-PDFF.
5. Understands the procedures and requirements of the study and provides written informed consent and authorization for protected health information disclosure.
6. Willing and able to comply with the requirements of the study protocol.

### Exclusion Criteria

Subjects Will be Excluded from the Study if any of the Following Criteria are Met:
1. Insulin-controlled diabetes.
2. Pregnant or breastfeeding or plans to become pregnant.
3. Intolerance to Magnetic Resonance Imaging (MRI) or with conditions contraindicated for MRI procedures including but not limited to inability to fit into MRI scanner or surgical clips/metallic implants/shrapnel. Subjects must not be claustrophobic, have a history of claustrophobia, or intolerance of closed or small spaces.
4. Weight gain or loss>5% in 3 months prior to study or >10% in 6 months prior to screening.
5. History of lap banding, intragastric balloon, duodenal-jejunal sleeve, or bariatric surgery within 5 years of screening, plans for bariatric surgery prior to conclusion of study participation, or plans to lose weight during this study either through a special diet, exercise program or both.
6. History of malignant hyperthermia.
7. History of chronic serious recurrent skin rashes of unknown cause.
8. History of or current clinically significant cardiovascular disease including but not limited to transient ischemic attack, stroke, cardiac arrhythmias, syncope, unstable angina, myocardial infarction in the 6 months prior to screening, congestive heart failure, or uncontrolled hypertension. (Uncontrolled hypertension is defined as a systolic blood pressure≥160 mmHg or a diastolic blood pressure≥100 mmHg based on an average of three resting determinations in the sitting position with an appropriately sized cuff).
9. Resting heart rate<45 or >110 bpm.
10. On screening ECG or by history:
   a. A marked baseline prolongation of QT/QTcF interval (e.g., repeated demonstration of a QTcF interval>450 msec for males and >470 msec for females).
   b. A history of additional risk factors for Torsades de Pointes (TdP) (e.g., heart failure, hypokalemia, family history of Long QT Syndrome) or a family history of sudden cardiac death of unknown origin.
11. Kidney disease, kidney transplant, or estimated glomerular filtration rate (eGFR)<50 mL/min/1.73 m² based on the CKD-EPI Creatinine Equation (NKF 2009; https://www.kidney.org/content/ckd-epi-creatinine-equation-2009).
12. Significant lung disease requiring chronic daily medication including chronic obstructive pulmonary disease (COPD), emphysema, pulmonary fibrosis, or asthma.
13. Untreated obesity hypoventilation syndrome (OHS) or obstructive sleep apnea (OSA).
14. History of or active (acute or chronic) liver disease other than nonalcoholic fatty liver disease (NAFLD)/nonalcoholic steatohepatitis (NASH), such as but not limited to autoimmune liver disease, viral hepatitis, genetic hemochromatosis, primary biliary cirrhosis, Wilson disease, alpha-1-antitrypsin deficiency, alcohol liver disease, acute fatty liver of pregnancy or drug-induced (including acetaminophen) liver disease.
15. History of or treatment for clinically significant gastroparesis, inflammatory bowel disease, or any surgery of the upper gastrointestinal tract with the exception of cholecystectomy, or minor gastric procedures that are approved by the medical monitor.
16. History of cirrhosis and/or hepatic decompensation, including ascites, hepatic encephalopathy, or variceal bleeding.
17. History of acute pancreatitis within one year of screening or chronic pancreatitis of any cause.
18. Serum triglyceride concentrations exceeding 500 mg/dL.
19. HbA_{1c}>9.0%.
20. Familial (mother/father/sibling) and/or personal history of retinal detachment any time in the past.
21. Any history of or current diagnosis of Glaucoma.
22. Evidence of the following on screening ophthalmologic examination:
   a. Peripheral retinal pathology requiring treatment, retinal tears, or lattice that require treatment.
   b. Diabetic retinopathy with macula exudates or macula edema as shown by optical coherence tomography (OCT) and examination.
   c. Any active macular disease that affects the vision, including macula pucker (epiretinal membrane) and macular degeneration.
   d. Visually significant cataract as determined by ophthalmologist.
   e. Any previous intravitreal injection of anti-VEGF agents for macular degeneration.
   f. History of prior vitrectomy.
23. History of malignant neoplasms within 5 years of screening, except for basal cell or squamous cell skin cancer, cervical carcinoma in situ, or prostate cancer that is not currently or expected to require radiation therapy, chemotherapy and/or surgical interventions or to initiate hormonal treatment.
24. History of organ transplantation.
25. Received a COVID-19 vaccine less than 1 week prior to dosing (Visit 2/Day 1) and/or plans to receive a COVID-19 vaccine during the study period.
26. History of significant drug abuse within one year prior to Screening or frequent use of soft drugs (such as marijuana) within 3 months prior to the Screening visit, or hard drugs (such as cocaine, phencyclidine [PCP], opioid derivatives including heroin, and amphetamine derivatives) within 1 year prior to screening.
27. History of alcoholism in the last 2 years or current evidence of excessive alcohol consumption as assessed by screening evaluation using the Alcohol Use Disorders Identification Test (AUDIT, Thompson 2018), and history of regular alcohol consumption exceeding approximately 14 drinks/week for men and 7 drinks/week for women [1 drink=4 ounces (120 mL) of wine or 12 ounces (360 mL) of beer or 1 ounce (30 mL) of hard liquor] within 6 months of Screening, as determined by the Investigator.
28. Positive urine drug screen for drugs of abuse or positive phosphatidylethanol (PEth) blood test result>100 ng/ml at Screening. In instances of an exclusionary PEth value, consideration for enrollment can be provided if the principal investigator and medical monitor agree the subject's history is not consistent with alcohol abuse.
29. Current regular vaping or more than 5 cigarettes or the equivalent per week. Use of nicotine patches for smoking cessation is permitted.
30. Positive test results of hepatitis B surface antigen (HBsAg), hepatitis C virus antibody (HCV Ab), or human immunodeficiency virus (HIV1/2) antibody.
31. Neutropenia, defined as absolute neutrophil count≤1000/μL.
32. Serum AST or ALT>5× upper limit of normal (ULN) at screening. (One repeat test may be allowed within 7 days at the discretion of the Investigator).
33. Total bilirubin>ULN, unless due to Gilbert's syndrome or if considered normal variability in the absence of other clinically relevant liver impairment as approved by Medical Monitor.
34. International normalized ratio (INR)≥1.3 at screening if there is other evidence of potential significant liver impairment.
35. Participation in another clinical trial at the time of screening or exposure to any investigational agent, including topical, within 30 days of screening or 5 half-lives, if half-life known.
36. No tattoo or body piercings during the course of the study. Any underlying physical or psychological medical condition that, in the opinion of the Investigator or sponsor, would make it unlikely that the subject is able comply with the study requirements or would be unable to complete the study.
37. Any condition that the investigator believes would interfere with his/her ability to provide written informed consent, comply with study instructions, or which might confound the interpretation of the study results or put the subject at undue risk.
38. Known or potential hypersensitivity to Compound 1 or its excipients.

### Prohibited Medications (Current Use):

39. Any herbal supplement, over the counter drug, mail order or prescription drug for weight loss.
40. Prescription or over the counter stimulants including: dextroamphetamine/Dexedrine, dextroamphetamine/amphetamine combination product/Adderall, or methylphenidate (Ritalin®, Concerta®).
41. Thiazolidinediones (TZD): pioglitazone/Actos, rosiglitazone/Avandia.
42. Glucagon-like peptide 1 (GLP1) agonists: exenatide/Byetta/Bydureon, lixisenatide/Adlyxin, liraglutide/Victoza, dulaglutide/Trulicity, semaglutide/Ozempic.
43. Sodium-glucose cotransporter-2 (SGLT2) inhibitors: canagliflozin/Invokana, dapagliflozin/Farxiga, empagliflozin/Jardiance, ertugliflozin/Steglatro.
44. Vitamin E: use of ursodiol or high dose vitamin E>400 IU/day for at least one month within in the last 6 months or started high dose vitamin E within last 3 months of screening.
45. Recent (within 3 months of screening) or current use of obeticholic acid/Ocaliva, systemic corticosteroids, methotrexate, tamoxifen, amiodarone, or long-term use of tetracyclines.
46. Warfarin, heparin, factor Xa inhibitors (dabigatran betrixaban edoxaban, apixaban, and rivaroxaban).
47. Concomitant medications that prolong the QT/QTc interval and are known to be associated with increased risk of Torsade des pointes as identified in the https://crediblemeds.org/website list category of ‘Known Risk’.
48. Products with cannabidiol (CBD).

### Treatment Plan and Methods

Study procedures should be completed as designated in the Schedule of Assessments (Table 4).

TABLE 4
Schedule of Assessments

| | | | | | | | | | | | Follow- | | |
|---|---|---|---|---|---|---|---|---|---|---|---|---|---|
| | | Treatment Visits | | | | | | | | | up | | |
| | Screening^{a} | | | Visit | Visit | Visit | Visit | Visit | Visit | Visit | Visit | | |
| | Visit 1 | Visit 2 | | 3 | 4 | 5 | 6 | 7 | 8 | 9^{b} | 10 | Early Termination Visit | |
| | Activity/Day | | | | | | | | | | | | |
| | Day −45 | Day 1: | Day 1: | Day | Day | Day | Day | Day | Day | Day | Day | <28 days | ≥28 days |
| | to −3 | Predose | Postdose | 2 | 7 | 14 | 28 | 42 | 49 | 61^{b} | 73 | of dosing | of dosing |
| | Visit Window | | | | | | | | | | | | |
| | | | | | ±1 | ±1 | ±4 | ±4 | ±2 | varies^{b} | ±2 | | |
| Informed Consent | X | | | | | | | | | | | | |
| Inclusion/Exclusion Criteria | X | X | | | | | | | | | | | |
| Medical History | X | X | | | | | | | | | | | |
| Alcohol use assessment | X | | | | | | | | | | | | |
| Blood PEth Test | X | | | | | | X | | | | | | |
| TSH and T4 | X | | | | | | | | | | | | |
| Serum triglycerides | X | | | | | | | | | | | | |
| HbA_{1c} | X | | | | | | | | | X | | X | X |
| Fibroscan | X | | | | | | | | | X | | X | X |
| Demographics | X | | | | | | | | | | | | |
| eGFR (CKD-EPI) | X | | | | | | | | | | | | |
| Coagulation (INR) | X | | | | | | | | | | | | |
| Serology: HIV, HBsAg, | X | | | | | | | | | | | | |
| HCV Ab | | | | | | | | | | | | | |
| Urine Drug | X | X | | | | | X | | | X | | | |
| Urine Pregnancy test | X | X | | | X | X | X | X | | X | X | X | X |
| (female only) | | | | | | | | | | | | | |
| Laboratory U/A (dipstick) | X | X | | | | | X | | | X | X | X | X |
| Hematology Panel (CBC) | X | X | | | | | X | | | X | X | X | X |
| Serum chemistry (includes | X | X | | | | | X | | | X | X | X | X |
| LFTs) | | | | | | | | | | | | | |
| Ophthalmologic exam | X | | | | | | | | | X | | | X |
| Vital signs^{c} and weight | X | X | X | X | X | X | X | X | X | X | X | X | X |
| (InBody scale) | | | | | | | | | | | | | |
| 12-lead ECG | X | X | | | X | X | X | X | | X | X | X | X |
| Concomitant medications | X | X | X | X | X | X | X | X | X | X | X | X | X |
| Adverse events | X | X | X | X | X | X | X | X | X | X | X | X | X |
| MRI/MRI-PDFF | X^{d} | | | | | | | | | | | | |
| Complete physical | X | | | | | | | | | X | | | X |
| (includes neuro exam: | | | | | | | | | | | | | |
| muscle strength) | | | | | | | | | | | | | |
| Targeted physical | | X | | | | X | X | X | | | X | X | |
| (includes neuro exam: | | | | | | | | | | | | | |
| muscle strength) | | | | | | | | | | | | | |
| Glycated Albumin | | X | | | | X | X | X | | X | | X | X |
| hs-CRP | | X | | | | | X | | | X | | | X |
| Lipid Panel (HDL, LDL, | | X | | | | | X | | | X | | | X |
| VLDL, FFA, cholesterol, | | | | | | | | | | | | | |
| triglycerides) | | | | | | | | | | | | | |
| ApoB and Lp(a) | | X | | | | | X | | | X | | | X |
| HOMA-IR: glucose, | | X | | | | | X | | | X | | | X |
| insulin, and C-peptide | | | | | | | | | | | | | |
| (3 draws 5 minutes apart) | | | | | | | | | | | | | |
| OWLiver (Metabolomics) | | X | | | | | X | | | X | | | X |
| OWL (Lipidomics) | | X | | | | | X | | | X | | | X |
| Proteomic Profile | | X | | | | | X | | | X | | | X |
| (SomaScan) | | | | | | | | | | | | | |
| Enhanced Liver Fibrosis | | X | | | | | | | | X | | | X |
| Assay (ELF) | | | | | | | | | | | | | |
| Exploratory biomarker | | X | | | | | X | | | X | | | X |
| (Serum sample) | | | | | | | | | | | | | |
| Exploratory biomarker | | X | | | | | X | | | X | | | X |
| (Plasma sample) | | | | | | | | | | | | | |
| Plasma sampling for PK^{e} | | X | X | X | X | X | X | X | | X | X | X | X |
| Dispense study drug | | | | X | X | X | X | X | X | | | | |
| Administer study drug | | X | | X | X | X | X | X | X | X | | | |
| Dispense Daily Log | | | | X | X | X | X | X | X | | | | |
| (dosing, temperature, diet) | | | | | | | | | | | | | |
| Collect Daily Log | | | | | X | X | X | X | X | X | | X | X |
| (dosing, temperature, diet) | | | | | | | | | | | | | |

^{a}Subjects will be screened over a 45 day period to determine their eligibility based on specific history, physical, laboratory and imaging evaluations as per the Schedule of Assessments. Due to scheduling of the procedures, multiple visits will likely be necessary to complete the screening process. However, if all screening assessments and procedures can be completed within 30 days of first dose, then a single screening visit is permissible.
^{b}Visit 9 may be split into a series of visits to complete all assessments.
^{c}Vital signs (body temperature, systolic and diastolic blood pressure, heart rate, and respiration rate) conducted twice (predose and prior to discharge) on Days 1, 14, and 28. Only predose vital sign assessments on other Treatment Visit Days.
^{d}By exception, MRI must be 30 days prior to Day 1 dosing.
^{e}PK Sampling: On Day 1, 14, and 28: predose, and approximately 2, 4, and 6 hours. On Days 2, 7, 42, and 61: predose sample. One sample during Visit 10 or Early Termination.

### Pharmacokinetic Parameters

The following PK parameters will be determined from concentration-time data:
Population PK (PPK) analysis of Compound 1 and 2,4-dinitrophenol. The following PK parameters will be calculated: Cₘₐₓ, Tₘₐₓ, t_{1/2}, T_{lag}, AUC₀₋₁, AUC_{0-∞}. CL/F, Vd/F, Az. Other PK parameters may be calculated, as data allow and appropriate.
As data permit, non-compartmental analysis of Compound 1 and 2,4-dinitrophenol. The following PK parameters will be calculated: Cₘₐₓ, AUC, and accumulation.

### Magnetic Resonance Imaging-Proton Density Fat Fraction (MRI-PDFF)

Magnetic Resonance Imaging Proton Density Fat Fraction (MRI-PDFF) is a non-invasive, quantitative biomarker to assess liver fat content (steatosis). The percentage of fat in the liver, or proton density fat fraction (PDFF), is being measured using MR: MRI-PDFF at baseline and end of treatment. Liver volume will be assessed from an axial T1 weighted or dual echo gradient echo images covering the entire liver. This advanced MRI technique measures the fraction of mobile protons in the liver attributable to liver fat (the PDFF), which is a direct measure of liver fat content and is a fundamental tissue property. Subjects will need to be fasting for 4 hours prior to the MRI-PDFF being performed.

### Abdominal MRI

An MRI image will be taken of the abdominal region to assess total fat. The two primary measurements from this scan will estimate the total visceral adipose tissue (VAT), the type of fat stored within the body cavity, and the subcutaneous adipose tissue (SAT), the type of fat visible right underneath the skin.

### Fibroscan®

The Fibroscan® is a non-invasive medical device which estimates liver fat content (steatosis) and liver stiffness (fibrosis). The assay works by measuring shear wave velocity. A 50-MHz wave is passed into the liver from a small transducer on the end of an ultrasound probe. The probe also has a transducer on the end that measure the velocity of the shear wave (in meters per second) as this wave passes through the liver. The shear wave velocity is converted into liver stiffness, which is expressed in kilopascals (VCTE score). A second measurement is also taken that estimates hepatic steatosis through measuring the ultrasonic attenuation of the echo wave, termed the controlled attenuation parameter (CAP). Subjects will need to be fasting for 4 hours prior to the Fibroscan® being conducted.

### One Way Liver Assays (OWL)

### OWL—Metabolomics

This metabolomics assay extracts metabolites from volunteer plasma and serum to take a snapshot of cellular function. Liquid chromatography-mass spectrometry (LC-MS) metabolomics is used to identify serum biomarkers that differentiate normal liver and NAFLD and between NASH and NAFLD. The metabolomics profile also provides insight into cellular function and inflammation through the examination of various cellular metabolites that provide insight into key molecular pathways.

### OWL—Lipidomics

Lipidomics is non-invasive blood assay that analyzes and identifies lipids in plasma and serum. These lipids will be separated and characterized via mass spectrometry and the analysis will include fatty acids, fatty acid derivatives, glycerolipids, glycerophospholipids, sphingolipids, and sterols.

### Slow Off-rate Modified Aptamer (SomaScan)

SomaScan is a non-invasive blood assay. Blood plasma and serum samples will be assayed using oligonucleotide aptamers whose three-dimensional conformational shape binds specifically to a protein target of interest. Over seven thousand proteins will be assayed and quantified, enabling a proteomic snapshot of the body. Different mathematical models have been applied to large clinical data to establish algorithms with predictive value in cardiovascular health, metabolic rate, lean body mass, liver inflammation, cardiorespiratory fitness, and glucose tolerance.

### Enhanced Liver Fibrosis (ELF)

The ELF assay is a non-invasive blood test that measures three markers of liver inflammation and fibrosis: hyaluronic acid, procollagen III amino-terminal peptide (PIIINP), and tissue inhibitor of matrix metalloproteinase 1 (TIMP-1). The values of these three markers, when used in conjunction with accompanying clinical data, are highly predictive of the inflammatory and fibrotic state of the liver, as evidenced by correlating the data with histology in larger clinical trials.

### Exploratory Biomarkers

Blood samples will be obtained and separated into plasma and serum components from which separate 400 μL aliquots will be drawn, labelled, and stored for future analysis of proteins, lipids, or gene expression that could assist in explaining the pharmacological actions of Compound 1.

### Ophthalmologic Examination

A full medical eye examination including fundus photographs of the posterior pole of eye, OCT of the maculas of both eyes, and slit lamp evaluations will be performed at screening and at the completion of dosing (approximately Day 61) to characterize the subject's baseline status and to monitor any changes from baseline over the course of treatment. Pupillary dilation will be accomplished with 2.5% neosynephrine and 0.5% tropicamide (Mydriacyl) (unless there is a contraindication deemed by the ophthalmologist), one drop in each eye one time in light color eyes and up to two times, 5 minutes apart in dark eyes. A slit lamp is a biomicroscope with a bright light used during an eye exam and assesses different structures at the front of the eye and inside the eye for determination of the health and detection of eye disease. OCT is a non-invasive imaging technique that uses light waves to take cross-section pictures of the retina. Fundus photography involves photographing the rear of the eye. These procedures are standard tests involved in the medical evaluation of the health of the eye and should be conducted by a limited number of coordinating ophthalmologists to ensure consistency of evaluations.

### Vital Signs

Vital signs include body temperature, systolic and diastolic blood pressure, heart rate, and respiration rate. All blood pressure readings must be done with a blood pressure cuff appropriate to the arm size of the subject. A blood pressure cuff that is too small will result in inaccurately high blood pressure determinations. Blood pressure and heart rate recordings will be made after the study subject has been supine for ≥5 minutes. Three blood pressures will be taken at each timepoint, with each blood pressure obtained approximately 2 minutes apart. The first blood pressure will be discarded. The second and third blood pressure measurements will be entered in the database, averaged, and will serve as the value for that timepoint.

The InBody scale will be used to capture weight, muscle, and body fat. This must be done predose, in the fasted state, and at approximately the same time of day.

Body temperature will also be monitored daily by the subject at home utilizing a Braun Thermoscan 7 inner ear thermometer which will be provided to them. The thermometer provides a color-coded display that displays the temperature as well as indicating normal, elevated (>99.9° F., yellow display), or fever (>103° F., red display) temperatures. There is an audible feedback system that ensures appropriate usage and alerts the user that the temperature has been acquired. Nine previous reading will be recorded on the thermometer. Temperature readings should be done daily at the time of dosing. Should the subject have symptoms that may indicate a fever, they should take their temperature and verify. For temperature elevations≥100° F., as indicated by the yellow or red display, the subject should stop taking Compound 1, avoid antipyretics (acetaminophen, aspirin or non-steroidal anti-inflammatory agents) and call the Investigational Site.

Clinical Laboratory Tests

### At Screening Only:

Viral serology will include testing for the presence of hepatitis B antigen, anti-hepatitis C antibody and anti-HIV antibodies.
TSH and Free T4.
eGFR=CKD-EPI Creatinine Equation (NKF 2009; www.kidney.org/content/ckd-epi-creatinine-equation-2009).

Hematology testing will include erythrocyte mean corpuscular hemoglobin concentration (MCHC), erythrocyte mean corpuscular volume (MCV), hematocrit, hemoglobin, leukocyte count, and absolute counts of lymphocytes, monocytes, neutrophils, basophils, eosinophils and platelets.

Serum chemistry analyses will include glucose, calcium, albumin, total protein, sodium, potassium, bicarbonate, chloride, magnesium, blood urea nitrogen (BUN), creatinine, alkaline phosphatase, phosphate, uric acid, lactate dehydrogenase, ALT, AST, gamma-glutamyl transferase (GGT), bilirubin (total and direct), amylase, and CPK.
Baseline ALT and AST=while each value will be recorded, the average of screening and Day 1 predose values will be used as the baseline value in the statistical evaluation of these parameters in the exploratory analysis.

Lipid panel will include total cholesterol, HDL, LDL, VLDL, triglycerides, and FFA.

Additional tests at select time points include glycated albumin, hs-CRP, ApoB, Lp (a), and HOMA-IR (includes glucose, insulin, and C-peptide; Wallace 2004), and PEth test.
For HOMA-IR, 3 blood samples for the 3 analytes (blood glucose, serum insulin, and C-peptide) will be drawn after a minimum of 5 minutes between each sample.
The PEth test is a serum biomarker that can assess recent alcohol consumption. The value is dependent on both quantity of alcohol and time from consumption. This will be assessed at screening and Day 28. At the discretion of the investigator, it can be obtained at other times if there is concern for excessive alcohol consumption based on history, symptoms or laboratory evaluations (e.g. elevated liver function tests).

Urinalysis will consist of dipstick evaluations, with a reflex microscopic evaluation if dipstick shows blood or protein is small (1+), moderate (2+) or large (3+). Spot urine protein and albumin to be done if urine>trace protein on 2 collections.

Urine pregnancy tests will be conducted for female subjects.

Laboratory tests may be repeated once at screening. Additional laboratory evaluation may be performed at the discretion of the investigator in the assessment of an adverse event, as medically warranted.

12-lead Electrocardiogram

Single 12-Lead ECG measurements will be obtained after the subject has rested in a supine position for at least 10 minutes. External stimuli should be kept to a minimum. Video games, watching of TV, and talking will not be allowed during this time. A digital ECG machine will be utilized for the trial. If an ECG timepoint coincides with any blood samples, the ECGs will performed #10 minutes from obtaining the blood sample at the same timepoint. In addition, whenever possible, subjects should not have a meal within 2 hours prior to an ECG being performed.

The ECGs will be measured using an ECG machine that automatically calculates the heart rate and measures PR, RR, QRS, QT, and QTcF (Fridericia correction formula). The same ECG machine should be used for the same subject throughout the study, if at all possible. ECGs should be conducted in adherence with a research unit SOP acceptable to the Sponsor.

### Example 6: Exploratory Phase 2A, Study of Compound 1 for the Treatment of Subjects With Obese Heart Failure With Preserved Ejection Fraction (HFpEF)

This is a Phase 2A, randomized, parallel-group, placebo-controlled, double-blind, within subject dose escalation trial with 3 dose levels of Comound 1 and placebo. Estimated 62 participants will be enrolled. Subjects will be randomized (1:1) either to Compound 1 or placebo. Two dose levels will be administered in sequential order (150 mg daily followed by 300 mg daily), each for 20 days, to reach the third and highest dose of 450 mg daily if safety and tolerability are demonstrated at the lower 2 preceding doses. Administration of the 450 mg high dose will continue for a total of 94 days, with a safety follow-up visit within-14 days of the last dose.

Subjects will be screened over a 40-day period to determine their eligibility based on specific history, physical, laboratory, and imaging evaluations as per the Schedule of Assessments. While a single screening clinical site visit is indicated, an additional visit may be necessary to complete the screening procedures due to scheduling issues. A number of these assessments will serve as the baseline prior to drug administration. A central laboratory will be used for all assessments, including MRI, DEXA, clinical blood/plasma measures, transthoracic echocardiography, and CPET.

Compound 1 is being evaluated for its efficacy in improving cardiovascular function in obese subjects with HF with preserved ejection fraction (HFpEF).

### Inclusion Criteria:

1. Adult male or female, >40 years of age.
2. Competent to understand the information given in the Institutional Review Board (IRB) or Independent Ethics Committee (IEC) approved Informed Consent Form (ICF) and must sign the form prior to the initiation of any study procedures.
3. Body mass index (BMI)>30 kg/m²;
4. Signs and symptoms of HF in the judgement of the Investigator, and meets the following disease severity criteria:
   a. KCCQ OSS<80; b. NYHA Classification Class II-III; c. Baseline peak VO₂<18 mL/kg/min for females or <20 mL/kg/min for males; d. Respiratory exchange ratio (respiratory quotient) (RER [RQ]) at baseline of >1.0; e. Left ventricular ejection fraction (EF)>50%; f. At least 1 of the following objective criteria for HF: i. Documented hospitalization with HF as primary cause within in last year, or if greater than the past year, then with addition of structural heart disease on echocardiography (increased left atrial volume size or left ventricular hypertrophy, with sex-specific cut-points as per Lang, 2015) as follows:
      Left ventricular hypertrophy (LVH):
   a. Men: Either septal wall thickness (cm) either>1.1 or posterior wall thickness>1.1;
b. Women: Either septal wall thickness (cm) either>1.0 or posterior wall thickness>1.0;
   Left atrial dilation (LAD): AP dimension (cm): >4.0 in men; >3.8 in women; ii. Pulmonary capillary wedge pressure (PCWP) at rest>15 mmHg (or left ventricular end-diastolic pressure [LVEDP]>18 mmHg) or >25 mmHg (or 2.0 mmHg/L/min) with exercise in the last year; iii. E/e′ ratio>14 at septal annulus at rest on Doppler and tissue Doppler imaging in the last year; or iv. Currently elevated NT-proBNP defined as >125 μg/ml without atrial fibrillation/flutter and >375 μg/mL for subjects with atrial fibrillation/flutter.
   5. Participants should maintain their stable level of physical activity throughout the duration of the study and must agree to not enroll in an exercise training program during the study.
   6. Participants should maintain their stable diet and no plan to enter into a weight loss program prior to or during the course of the study.
   7. Euthyroid as assessed by a thyroid profile utilizing thyroid stimulating hormone (TSH) and free thyroxine (T4) testing at screening. Subjects with a stable history of thyroid disease and who have been on stable doses of thyroid medications for a minimum of 4 months can be enrolled.
   8. Ambulatory (not wheelchair- or scooter-dependent) and able to perform upright exercise testing including a 6 MWT.
   9. Stable doses of medications (defined as no new medication or change in existing dose of medication>50%) for 30 days prior to screening, with additional specific criteria for the diuretics:
      a. If treated with a loop or thiazide diuretic, must be on stable regimen, which dose permits a flexible diuretic dosing schedule.

### Exclusion Criteria:

1. Life expectancy<1 year due to non-cardiovascular reasons, in the judgement of the Investigator.
2. History of malignancy within 5 years (except non-high-grade skin cancers, carcinoma-in-situ, or low-grade prostate cancer).
   3. Weight change (gain or loss) of ≥10 pounds either by self-reporting or documented weight loss within the past 90 days.
   4. Bariatric surgery prior to screening or planned bariatric surgery during the course of the study.
   5. Treatment with GLP-1 receptor antagonist begun within 1 year of screening.
   6. Treatment with SGLT2 inhibitors begun within 6 months of screening.
   7. Intolerance to MRI or with conditions contraindicated for MRI procedures including but not limited to:
      a. Having surgical clips/metallic implants/shrapnel/internal electric implants; or
      b Inability to fit into MRI scanner due to subject habitus or exceeding weight tolerance limit of the scanner (generally, 350 or 400 lbs, dependent on manufacturer); or
      c. Claustrophobia: history of severe claustrophobia that would lead to inability to conduct MRI.
   8. Current acute decompensated HF requiring intravenous (IV)diuretics or recent (<1 month before screening) hospitalization for HF.
   9. Primary cardiomyopathy (e.g., constrictive, restrictive, infiltrative, toxic, hypertrophic [congenital], congenital, or any other primary cardiomyopathy, in the judgement of the Investigator.
   10. Active myocarditis (COVID-induced or otherwise).
   11. Active collagen vascular disease.
   12. Current greater than moderate left- or right sided valve disease, in the opinion of the Investigator.
   13. Planned cardiac surgery or catheter intervention during the time of trial participation.
   14. Prior documented EF<40% within the last 3 years.
   15. Tachycardia (>110 beats/minute) at screening.
   16. Atrial fibrillation or atrial flutter with an uncontrolled heart rate response or with a resting heart rate greater than 110 bpm by ECG at screening. Subjects may rescreen after appropriate adjustment of medication to manage the atrial fibrillation. A maximum of 16 subjects with this condition can be enrolled in this study.
   17. Untreated, life-threatening dysrhythmia.

### Phase 2a Trial Results

The phase 2a metabolic trial of Compound 1 was a 61-day randomized, double-blind, placebo-controlled trial designed to assess the safety and efficacy of three dose levels of Compound 1 (150 mg, 300 mg, and 450 mg) in obese participants (body mass index 28 to 45 kg/m²) with elevated liver fat (greater than 8%). Eighty (80) participants ranging in age between 28 and 65 years were randomly assigned to one of three Compound 1 treatment groups or the matched placebo group, stratified and blocked for HbA1C levels of 5.7% or greater, and dosed once daily (fasting). Participants were instructed to not change behavior with regard to diet or exercise. The Phase 2a trial met primary (liver fat reduction by MRI-PDFF) and secondary (body weight and fat reduction by abdominal MRI) endpoints. Key results and observations include:
Statistically significant (p<0.0001 by ANCOVA) reductions in liver fat at all three dose levels.
   Relative reductions in liver fat were 33%, 43%, and 40% corresponding to responder rates (>30% relative reduction) of 40%, 71% and 72% at low, mid and high doses, respectively. Placebo relative reduction of liver fat of 2% and responder rate of 5%.
Key cardiovascular and metabolic health indicators observed across all dosing levels include:
   Dose dependent reduction in glycated albumin, an indicator of glucose control and insulin function (p<0.0001, high dose).
   Dose dependent reduction in inflammation marker high sensitivity C-reactive protein (hsCRP), an important parameter of cardiovascular risk (p<0.005, high dose).
Compound 1 was well-tolerated at all dose levels with excellent compliance. No Serious Adverse Events or deaths were reported. Diarrhea and transient flushing associated with alcohol intake, occurring in 25% and 31.6% of Compound 1 subjects respectively, were the most commonly reported Treatment Emergent Adverse Events. The majority of these events were mild; one participant discontinued Compound 1 for diarrhea in the low dose arm while no participant discontinued for any reason at the high dose.

Specific efficacy and safety of Phase 2a results are shown in Figures and Tables below:
(1). Compound I is well tolerated over eight weeks as shown in Table 5.

TABLE 5

| | CM 1 Daily Dose | | | | |
|---|---|---|---|---|---|
| | 150 mg | 300 mg | 450 mg | All CM1 | Placebo |
| | (N = 20) | (N = 21) | (N = 19) | (N = 60) | (N = 20) |
| Category | n (%) | n (%) | n (%) | n (%) | n (%) |

Number (%) of subjects with at least 1:

| Treatment-emergent adverse event (TEAE) | 13 | (65.0) | 17 | (81.0) | 16 | (84.2) | 46 | (76.7) | 11 | (55.0) |
|---|---|---|---|---|---|---|---|---|---|---|
| Severe TEAE | 1 | (5.0) | 0 | | 0 | | 1 | (1.7) | 0 | |
| Treatment-related TEAE^{a} | 9 | (45.0) | 16 | (76.2) | 14 | (73.7) | 39 | (65.0) | 7 | (35.0) |
| Fatal event (death) | 0 | | 0 | | 0 | | 0 | | 0 | |
| Serious TEAE | 0 | | 0 | | 0 | | 0 | | 0 | |
| TEAE leading to withdrawal of study drug | 2 | (10.0) | 1 | (4.8) | 0 | | 3 | (5.0) | 2 | (10.0) |
| Treatment-related TEAE of maximum severity | | | | | | | | | | |
| Mild | 5 | (25.0) | 10 | (41.6) | 11 | (57.9) | 26 | (43.3) | 6 | (30.0) |
| Moderate | 3 | (15.0) | 6 | (28.6) | 3 | (15.8) | 12 | (20.0) | 1 | (5.0) |

| | | | | | | | |
|---|---|---|---|---|---|---|---|
| Severe | 1 | (5.0) | 0 | 0 | 1 | (1.7) | 0 |

Number of adverse events:

| | | | | | |
|---|---|---|---|---|---|
| All TEAEs | 42 | 53 | 54 | 149 | 32 |
| Treatment-related TEAEs^{a} | 24 | 37 | 34 | 95 | 13 |
| Serious TEAEs | 0 | 0 | 0 | 0 | 0 |

^{a}Treatment-related adverse events are those that the investigator assessed as possibly or probably related to the study treatment

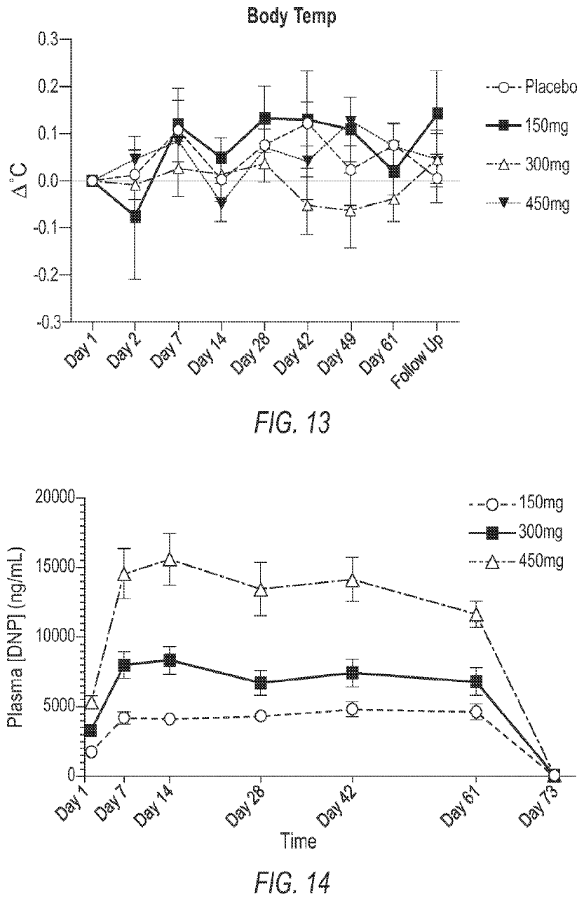

(2) Body temperature changes as shown in FIG. 13 and Table 6.

TABLE 6
Study Day

| | | P value | Statistically |
|---|---|---|---|
| Fixed effects (type III) | P value | summary | significant (P < 0.05)? |
| Time | 0.1543 | ns | No |
| Treatment | 0.4318 | ns | No |
| Time × Treatment | 0.7274 | ns | No |

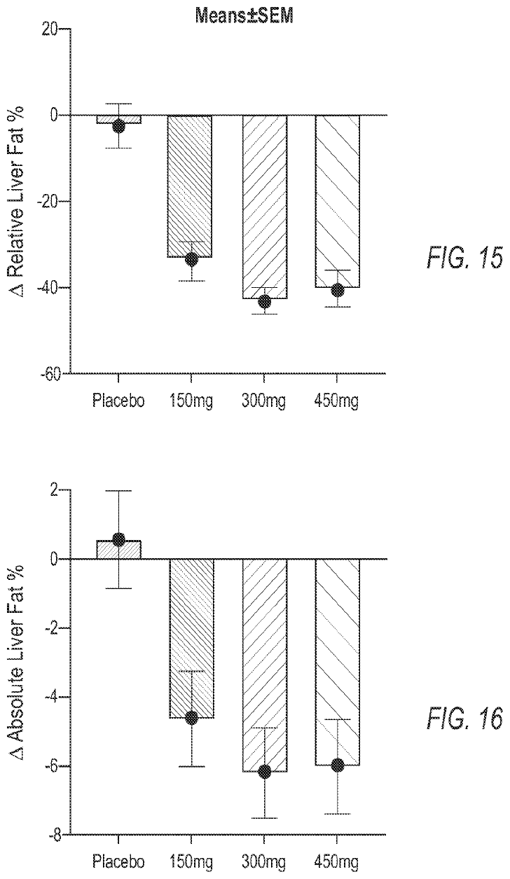

(3) PK result (Mean±SEM) as shown FIG. 14.
(4) Treatment effect demonstrated across all doses as shown in FIG. 15 and Table 7.

Placebo-Corrected percent change from baseline values for MRI-proton Density fat fraction (PDFF).

TABLE 7

| | | CM1 150 mg | CM1 300 mg | CM1 450 mg | Placebo |
|---|---|---|---|---|---|
| Visit | Statistic | (N = 20) | (N = 21) | (N = 18) | (N = 20) |
| Change from | n | 18 | 20 | 18 | 17 |
| Baseline | Mean | −33.389 | −42.958 | −40.369 | −2.374 |
| \|to Day 61 | SD | 17.5340 | 14.0958 | 17.5982 | 20.8430 |
| | Median | −32.830 | −41.400 | −42.335 | 0.210 |
| | Min, Max | −57.60, 11.08 | −74.74, −12.56 | −64.54, 7.33 | −37.87, 30.57 |

Baseline is the last non-missing value prior to the first dose of study medication (5) Treatment effect demonstrated across all doses as shown in FIG. 16 and Table 8.

Analysis of covariance for MRI-proton density fat fraction (PDFF). Mean change from baseline at Day 61 of FAS population (LSMean±95%)

TABLE 8

| | | | CM1 300 mg | CM1 450 mg | Placebo |
|---|---|---|---|---|---|
| Visit | Statistic | CM1 150 mg | (N = 20) | (N = 18) | (N = 20) |
| Day 61 | LS Mean | −4.64 | −6.21 | −6.03 | 0.57 |
| | 95% CI of LS Mean | (−6.03, −3.25) | (−7.52, −4.90) | (−7.40, −4.66) | (−0.85, 1.99) |
| | Difference of LS Mean and Placebo | −5.21 | −6.77 | −6.60 | |
| | 95% CI of Difference of LS Means | (−7.19, −3.23) | (−8.69, −4.86) | (−8.56, −4.64) | |
| | 2-sided P-value | <.0001 | <.0001 | <.0001 | |

Note:
This analysis was performed with the model including treatment as a fixed effect and baseline HbA1c stratification as a factor with baseline value of the response variable as a covariate.
Note:
LS = Least Squares,
CI = Confidence Interval |

(6) Treatment results in response (>30% liver fat reduction by MRI-PDFF) across all dose arms as show in Table 9.

TABLE 9

| Responder Status | CM1 150 mg | | CM1 300 mg | | CM1 450 mg | | All CM1 | | Placebo | |
|---|---|---|---|---|---|---|---|---|---|---|
| (FAS Population) | (N = 20) | | (N = 21) | | (N = 18) | | (N = 59) | | (N = 20) | |
| Responder | 8 | (40.0%) | 15 | (71.4%) | 13 | (72.2%) | 36 | (61.0%) | 1 | (5.0%) |
| Non-responder | 10 | (50.0%) | 5 | (23.8%) | 5 | (27.8%) | 20 | (33.9%) | 16 | (80.0%) |
| Responder Status | 150 mg CM1 | | 300 mg CM1 | | 450 CM1 | | All CM1 | | Placebo | |
| (HbA1c 5.7%-9.0%) | (N = 7) | | (N = 8) | | (N = 7) | | (N = 22) | | (N = 7) | |
| Responder | 3 | (42.9%) | 6 | (75.0%) | 6 | (85.7%) | 15 | (68.2%) | 0 | (0.0%) |
| Non-responder | 3 | (42.9%) | 1 | (12.5%) | 1 | (14.3%) | 5 | (22.74%) | 6 | (85.7%) |

MRI-PDFF response rate at Day 61 450 mg and 300 mg comparable >300 mG

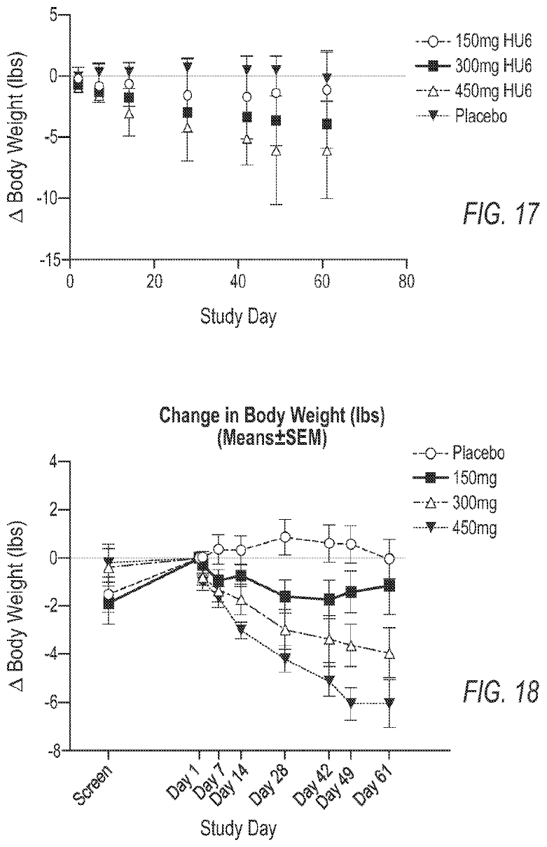

(7) Significant weight reduction at Compound 1 doses of 300 mg and 450 mg once daily as shown in FIG. 17 and Table 10.

Repeated measures analysis for InBody body weight. Mean change from baseline FAS population (LSMean±95% CI).

TABLE 10

| | | CM1 150 mg | CM1 300 mg | CM1 450 mg | Placebo |
|---|---|---|---|---|---|
| Visit | Statistic | (N = 20) | (N = 21) | (N = 18) | (N = 20) |
| Day 61 | LS Mean (SEM) | −1.14 (1.028) | −3.92 (0.981) | −6.04 (1.034) | −0.21 (1.049) |
| | 95% CI of LS Mean | (−3.19, 0.91) | (−5.87, −1.96) | (−8.10, −3.98) | (−2.30, 1.88) |
| | Difference of LS Mean and Placebo (SEM) | −0.93 (1.467) | −3.71 (1.435) | −5.83 (1.471) | |
| | 95% CI of Difference of LS Means | (−3.86, 1.99) | (−6.57, −0.85) | (−8.77, −2.90) | |
| | 2-sided P-value | 0.5266 | 0.0117 | 0.0002 | |

(8) Body weight observed as shown in FIG. 18 and Table 11.

TABLE 11
Day 61

| | | | | | |
|---|---|---|---|---|---|
| Placebo vs. 150 mg | 1.131 | −1.302 to 3.564 | No | ns | 0.4362 |
| Placebo vs. 300 mg | 3.904 | 1.665 to 6.142 | Yes | ** | 0.0057 |
| Placebo vs. 450 mg | 6.014 | 3.820 to 8.209 | Yes | **** | <0.0001 |

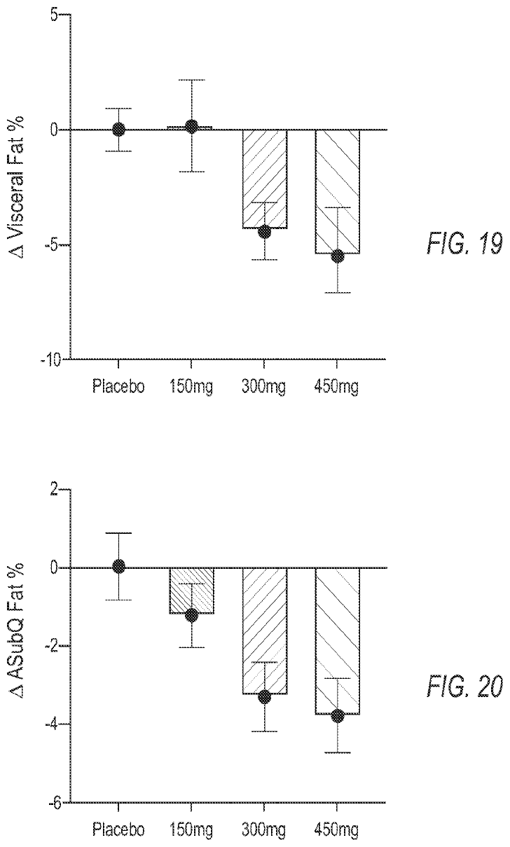

(9) Visceral adipose tissue-observed data FAS population as shown in FIG. 19 and Table 12.

Placebo-corrected percent change from baseline values for abdominal MRI liver volume and adiposity by treatment group FAS population (Mean±SEM).

TABLE 12

| Uncorrected Fisher's LSD | Mean Diff. | 95.00% CI of diff. | Below threshold? | Summary | Individual P Value |
|---|---|---|---|---|---|
| Placebo vs. 150 mg | −0.1480 | −4.864 to 4.568 | No | ns | 0.9503 |
| Placebo vs. 300 mg | 4.442 | −0.1583 to 9.042 | No | ns | 0.0582 |
| Placebo vs. 450 mg | 5.513 | 0.7968 to 10.23 | Yes | * | 0.0226 |

(10) Subcutaneous adipose tissue observed data FAS population as shown in FIG. 20 and Table 13.

Placebo-corrected percent change from baseline values for abdominal MRI liver volume and adiposity by treatment group FAS population (Mean±SEM).

TABLE 13

| Uncorrected Fisher's LSD | Mean Diff. | 95.00% CI of diff. | Below threshold? | Summary | Individual P Value |
|---|---|---|---|---|---|
| Placebo vs. 150 mg | 1.237 | −1.331 to 3.805 | No | ns | 0.3398 |
| Placebo vs. 300 mg | 3.327 | 0.8203 to 5.834 | Yes | * | 0.0100 |
| Placebo vs. 450 mg | 3.811 | 1.243 to 6.379 | Yes | ** | 0.0042 |

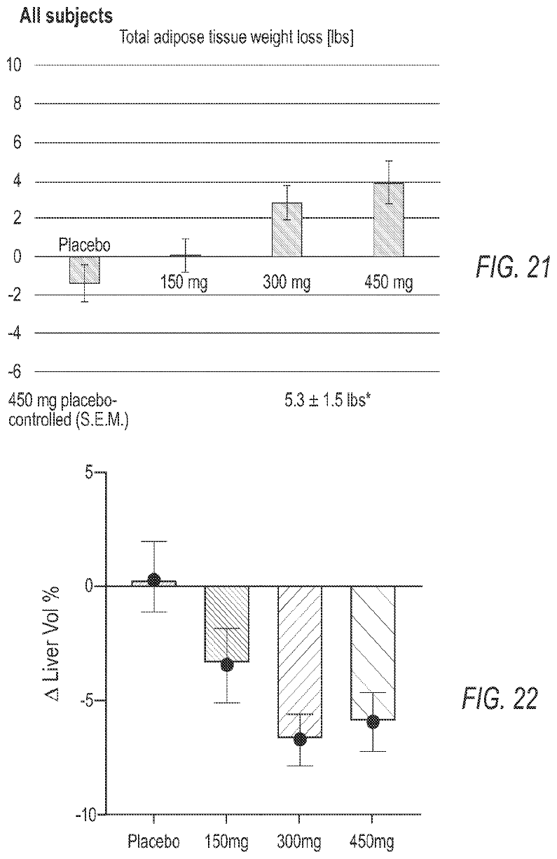

(11) Confirmation of fat loss (total adipose tissue) by MRI as shown in FIG. 21.
(12) Liver volume observed data FAS population as shown in FIG. 22 and Table 14.

TABLE 14

| Uncorrected Fisher's LSD | Mean Diff. | 95.00% CI of diff. | Below threshold? | Summary | Individual P Value |
|---|---|---|---|---|---|
| Placebo vs. 150 mg | 3.669 | −0.5255 to 7.864 | No | ns | 0.0854 |
| Placebo vs. 300 mg | 6.949 | 2.858 to 11.04 | Yes | ** | 0.0012 |
| Placebo vs. 450 mg | 6.173 | 1.978 to 10.37 | Yes | ** | 0.0045 |

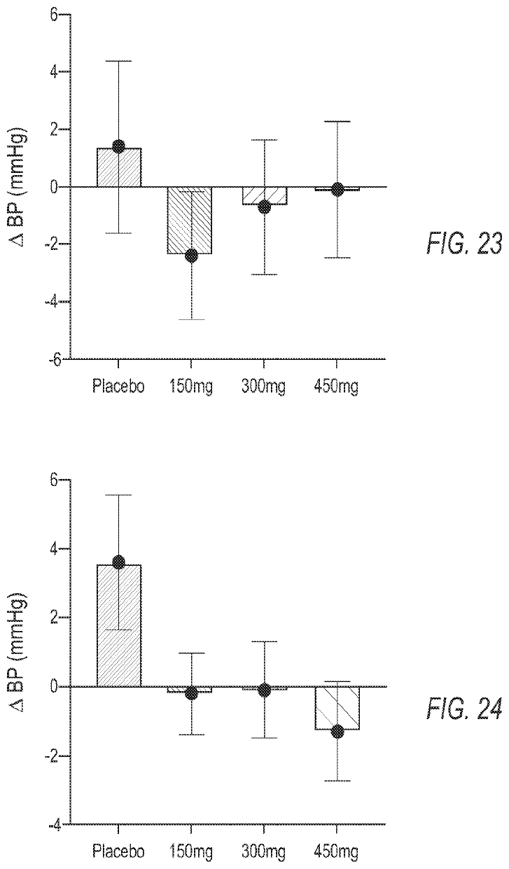

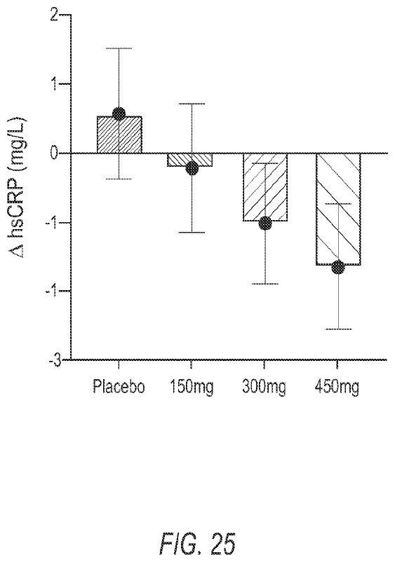

(13) Observed systolic blood pressure as shown in FIG. 23.
Mean change from baseline at Day 61 FAS population (Mean±SEM)
(14) Observed diastolic blood pressure as shown in FIG. 24.
Mean change from baseline at Day 61 FAS population (Mean±SEM)
(15) Observed High Sensitivity C-reactive protein (hsCRP) as shown in FIG. 25 and Table 15.
Mean change from baseline at Day 61 FAS population (LSMean±95CI)

TABLE 15

| | | CM1 150 mg | CM1 300 mg | CM1 450 mg | Placebo |
|---|---|---|---|---|---|
| Visit | Statistic | (19-20) | (N-21) | (N-18) | (N-20) |
| Day 61 | LS Mean (SEM) | −0.22 (0.464) | −1.02 (0.440) | −1.65 (0.460) | 0.57 (0.472) |
| | 95% CI of LS Mean | (−1.14, 0.71) | (−1.90, −0.15) | (−2.56, −0.73) | (−0.37, 1.52) |
| | Difference of LS Mean and Placebo | −0.79 (0.656) | −1.60 (0.640) | −2.22 (0.655) | |
| | 95% CI of Difference of LS Means | (−2.10, 0.52) | (−2.88, −0.32) | (−3.53, −0.91) | |
| | 2-sided P-value | 0.2303 | 0.0149 | 0.0012 | |

1-7. (canceled)

8. A method for treating heart failure with preserved ejection fraction (HFpEF), heart failure with reduced ejection fraction (HFrEF), or heart failure with mid-range ejection fraction (HFmrEF) in a subject, comprising administering to the subject a therapeutically effective amount of 5-[(2,4-dinitrophenoxy)methyl]-1-methyl-2-nitro-1H-imidazole, or a pharmaceutically acceptable salt thereof.

9. The method of claim 8, wherein the subject is suffering from at least one of the symptoms selected from shortness of breath, shortness of breath with exertion, impaired energetics in the heart, dizziness, fatigue, dyspnea, palpitations (atrial fibrillation), chest discomfort, edema, syncope, a limit on an activity of daily living, reduced exercise tolerance, fatigue, tiredness, increased time to recover after exercise, ankle swelling, coronary artery disease, hypertension, and heart murmur.

10-12. (canceled)

13. The method of claim 8, wherein the subject experiences an improvement of cardiac bioenergetic deficiency after administration, wherein the improvement comprising: a) weight loss>5%, b) reduction in blood pressure, and/or c) a reduction in the risk of a major cardiovascular event, wherein the major cardiovascular event is selected from the group consisting of death, hospitalization for worsening of the disease, and myocardial infraction.

14. The method of claim 8, further comprising assessing peak oxygen consumption (VO₂) and/or VE/CO₂ or VE/VCO₂ slope in the subject during exercise before and after administration of the therapeutically effective amount of 5-[(2,4-dinitrophenoxy)methyl]-1-methyl-2-nitro-1H-imidazole, wherein an increase in VO₂ in the subject after administration indicates a reduction in the extent of HFpEF, HFrEF, HFmrEF in the subject.

15. (canceled)

16. The method of claim 8, further comprising assessing 6-minute walk distance (6MWD) in the subject during exercise before and after administration of the therapeutically effective amount of 5-[(2,4-dinitrophenoxy)methyl]-1-methyl-2-nitro-1H-imidazole, wherein an increase in 6MWD in the subject after administration indicates a reduction in the extent of HFpEF, HFrEF, HFmrEF in the subject.

17. (canceled)

18. The method of claim 8, wherein the treatment further comprises assessing a NYHA classification score of the subject before and after administration, wherein a decreased NYHA score after administration indicates a reduction in the extent of the cardiac disease in the subject.

19-30. (canceled)

31. The method of claim 8, wherein the subject suffers from obesity, excess body fat, diabetes, high blood pressure (hypertension), dyslipidemia, hypertriglyceridemia, acquired lipodystrophy, inherited lipodystrophy, partial lipodystrophy, or metabolic syndrome.

32. A method of treating a cardiovascular disease comprising administering a therapeutically effective amount of 5-[(2,4-dinitrophenoxy)methyl]-1-methyl-2-nitro-1H-imidazole or a pharmaceutically acceptable salt thereof in a subject, to achieve at least one of:
i) a steady state of maximum plasma concentration (Cₘₐₓ) of 2,4-dinitrophenol from about 80 ng/ml to about 8300 ng/ml;
ii) mean half-life (t_{1/2}) of 2,4-dinitrophenol about 20-50 hours, about 25-40 hours, or about 30-40 hours;
iii) median time to maximum plasma concentration (Tₘₐₓ) of 2,4-dinitrophenol about 6-8 hours or about 6-10 hours;
iv) median area under the curve extrapolated to infinity (AUC_{inf}) of 2,4-dinitrophenol about 3 h*μg/mL to about 420 h*μg/mL; and
v) AUC/Cₘₐₓ ratio of about 18.

33. The method of claim 8, wherein the method does not cause a clinically significant risk of adverse events after administration.

34. The method of claim 33, wherein the adverse events comprise at least one of nausea, vomiting, sweating, dizziness, headaches, cataracts, glaucoma, pyrexia, hyperthermia, tachycardia, diaphoresis, tachypnoea, and death.

35. The method of claim 34, wherein the adverse event is characterized by at least one of elevated body temperature, elevated heart rate, abnormal sweating, erythema, perspiration, dehydration, and abnormally rapid breathing.

36-129. (canceled)

130. A method of reducing (a) liver fat by at least 50% or (b) lipids by at least 10% in a subject, comprising administering to the subject a therapeutically effective amount of 5-[(2,4-dinitrophenoxy)methyl]-1-methyl-2-nitro-1H-imidazole, or a pharmaceutically acceptable salt thereof.

131. The method of claim 130, wherein the subject is suffering from non-alcoholic fatty liver disease (NAFLD), non-alcoholic steatohepatitis (NASH), and/or hepatic steatosis.

132-135. (canceled)

136. The method of claim 130, wherein the subject suffers from at least one of obesity, excess body fat, type 2 diabetes, insulin resistance or intolerance, high blood pressure, dyslipidemia, atherosclerosis, hypertriglyceridemia, acquired lipodystrophy, inherited lipodystrophy, partial lipodystrophy, metabolic syndrome, Rett's syndrome, metabolic syndrome associated with aging, metabolic diseases associated with increased reactive oxygen species (ROS), Friedreich's ataxia, NAFLD, NASH, noncirrhotic NASH, noncirrhotic NASH with liver fibrosis, hepatic steatosis, hepatic fibrosis, liver cirrhosis, and hepatocellular carcinoma.

137. (canceled)

138. The method of claim 130, wherein the method provides at least one of:
i) a reduction of body weight by at least 10%;
ii) a reduction of HbA_{1c} by at least 0.5%, or at least 1.5%;
iii) a reduction of blood pressure of at least 5 mmHg;
iv) a reduction of lipids by at least 10%;
v) a reduction of liver fat by at least 50%;
vi) a reduction of serum alanine aminotransferase (ALT); and
vii) a reduction of aspartate aminotransferase (AST).

139. The method of claim 130, wherein the method does not cause a clinically significant risk of adverse events in the subject after administration.

140-145. (canceled)

146. The method of claim 130, wherein the method provides at least one of the following in the subject after administering about 30 mg to about 1400 mg of 5-[(2,4-dinitrophenoxy)methyl]-1-methyl-2-nitro-1H-imidazole:
i) a steady state of maximum plasma concentration (Cₘₐₓ) of 2,4-dinitrophenol from about 80 ng/mL to about 8300 ng/mL;
ii) mean half-life (t_{1/2}) of 2,4-dinitrophenol about 20-50 hours, about 25-40 hours, or about 30-40 hours;
iii) median time to maximum plasma concentration (Tₘₐₓ) of 2,4-dinitrophenol about 6-8 hours or about 6-10 hours;
iv) median area under the curve extrapolated to infinity (AUC_{inf}) of 2,4-dinitrophenol about 3 h*μg/mL to about 420 h*μg/mL; and
v) AUC/Cₘₐₓ ratio of about 18.

147. A method of treating or reducing the risk of cancer in a subject, comprising administering to the subject a therapeutically effective amount of 5-[(2,4-dinitrophenoxy)methyl]-1-methyl-2-nitro-1H-imidazole, or a pharmaceutically acceptable salt thereof.

148-151. (canceled)

152. The method of claim 130, wherein the subject has BMI of about 28.0 kg/m² to about 45.0 kg/m².

153-155. (canceled)

156. The method of claim 8, wherein the therapeutically effective amount is from about 30 mg to about 1400 mg per day, from about 100 mg to about 1000 mg per day, from about 150 mg to about 600 mg per day, or from 200 mg to 550 mg per day.

157-160. (canceled)

161. The method of claim 156, wherein the therapeutically effective amount is about 150 mg, 300 mg, 450 mg per day.

162. The method of claim 8, wherein 5-[(2,4-dinitrophenoxy)methyl]-1-methyl-2-nitro-1H-imidazole is administered orally once daily.

163. The method of claim 8, wherein 5-[(2,4-dinitrophenoxy)methyl]-1-methyl-2-nitro-1H-imidazole is administered once daily for two weeks, four weeks, six weeks, eight weeks, ten weeks, one month, two months, three months, four months, six months, eight month, or one year.

164. (canceled)
